(19) United States
(12) Patent Application Publication
(10) Pub. No.: US 2023/0326549 A1
(43) Pub. Date: Oct. 12, 2023

(54) COPY NUMBER VARIANT CALLING FOR LPA KIV-2 REPEAT

(71) Applicant: Illumina, Inc., San Diego, CA (US)

(72) Inventors: Michael A. Eberle, San Diego, CA (US); Jonathan Robert Belyeu, San Diego, CA (US); Xiao Chen, San Diego, CA (US)

(21) Appl. No.: 18/193,206

(22) Filed: Mar. 30, 2023

Related U.S. Application Data
(60) Provisional application No. 63/325,930, filed on Mar. 31, 2022.

(51) Int. Cl. G16B 20/10; G16B 30/10; G16B 20/20; G16H 50/30

(52) U.S. Cl. CPC G16B 20/10; G16B 30/10; G16B 20/20; G16H 50/30

(57) ABSTRACT

Disclosed herein include systems, devices, and methods for determining the total copy number of kringle IV type 2 (KIV-2) domain of LPA gene, and/or the copy number of KIV-2 domain of each allele of LPA gene, a subject from sequence reads (e.g., short reads) generated from a sample obtained from the subject.

### CROSS-REFERENCE TO RELATED APPLICATIONS

This application claims the benefit under 35 U.S.C. § 119(e) to U.S. Provisional Patent Application No. 63/325,930, filed Mar. 31, 2022. The content of this related application is incorporated herein by reference in its entirety for all purposes.

### REFERENCE TO SEQUENCE LISTING

The present application is being filed along with a Sequence Listing in electronic format. The Sequence Listing is provided as a file entitled 47CX-311982-US_SequenceListing, created Mar. 29, 2023, which is 8 kilobytes in size. The information in the electronic format of the Sequence Listing is incorporated herein by reference in its entirety.

### BACKGROUND

### Field

The present disclosure relates generally to the field of processing sequencing data, and more particular to determining a copy number of kringle IV type 2 (KIV-2) domain of LPA gene.

### Description of the Related Art

Understanding the genomic complexity of adult diseases such as cardiovascular disease (CVD) is the next frontier in genomics. Much of a person's risk of CVD is genetically predetermined, but can be circumvented with proper treatment and lifestyle changes. One of the clearest relations of gene to protein to disease for coronary heart disease (CHD) is Lipoprotein(a) (Lp(a)). There is a need for a short-read copy number (CN) caller that can determine the total number of copies of the KIV-2 repeat.

### SUMMARY

Disclosed herein include methods of determining a copy number of kringle IV type 2 (KIV-2) domain of LPA gene. In some embodiments, a method of determining a copy number of KIV-2 domain of LPA gene is under control of a processor (e.g., a hardware processor or a virtual processor) and comprises: receiving a plurality of sequence reads generated from a sample obtained from a subject (which can be a mammal, such as a human). The method can comprise: aligning the plurality of sequence reads to a reference genome sequence, comprising one or more copies of the KIV-2 domain of the LPA gene, to obtain a plurality of aligned sequence reads comprising sequence reads aligned to any copy of the KIV-2 domain of the LPA gene in the reference genome sequence. The method can comprise: determining a number of the sequence reads aligned to any copy of the KIV-2 domain of the LPA gene in the reference genome sequence. The method can comprise: determining a number of copies of a region of the LPA gene comprising the one or more copies of the KIV-2 domain based on the number of the sequence reads aligned to any copy of the KIV-2 domain of the LPA gene in the reference genome sequence. The method can comprise: determining a total copy number of the KIV-2 domain of the LPA gene of the subject using (a) the number of copies of the region of the LPA gene comprising the one or more copies of the KIV-2 domain and (b) a number of copies of the KIV-2 domain of the LPA gene in the reference genome sequence.

In some embodiments, the method further comprises: determining (a) a number of copies of the KIV-2 domain of the LPA gene of a first allele of the subject and (b) a number of copies of the KIV-2 domain of the LPA gene of a second allele of the subject, based on one or more single nucleotide variants (SNVs) of the KIV-2 domain of the LPA gene. In some embodiments, the one or more SNVs comprise T>G at position 296 and C>G at position 1264 of a copy of the KIV-2 domain of the LPA gene in the reference genome sequence. The copy of the KIV-2 domain can comprise a sequence of SEQ ID NO: 1. The one or more SNVs comprise T>G at chr6:160630428, 160635977, 160641520, 160624884, 160619338, and/or 160613786 of hg38 and/or C>G at chr6:160620306, 160625852, 160631396, 160636945, 160642488, and/or 160614754 of hg38 or at corresponding positions of another reference genome sequence.

In some embodiments, a sequence read of the sequence reads aligned to any copy of the KIV-2 domain of the LPA gene in the reference genome sequence with a low alignment quality score. In some embodiments, the number of the sequence reads aligned to any copy of the KIV-2 domain of the LPA gene in the reference sequence comprises a raw number or a normalized and/or GC-corrected number of the sequence reads aligned to any copy of the KIV-2 domain of the LPA gene in the reference sequence.

In some embodiments, determining the number of copies of the region of the LPA gene comprising the one or more copies of the KIV-2 domain comprises: determining the number of copies of the region of the LPA gene comprising the one or more copies of the KIV-2 domain using a normalized and/or GC-corrected number of the sequence reads aligned to any copy of the KIV-2 domain of the LPA gene in the reference genome sequence. In some embodiments, the method comprises: determining the normalized number of the sequence reads aligned to any copy of the KIV-2 domain of the LPA gene in the reference genome sequence using (1a) a depth of the sequence reads aligned to any copy of the KIV-2 domain of the LPA gene in the reference genome sequence, (1b) a length of the region of the LPA gene in the reference genome sequence comprising the one or more copies of the KIV-2 domain, (2a) a depth of sequence reads of the plurality of sequence reads aligned to each of a plurality of regions of the reference genome sequence other than a genetic locus comprising LPA gene, and (2b) a length of each of the plurality of regions of the reference genome other than the genetic locus comprising LPA gene. In some embodiments, the method further comprises: determining the GC corrected number of the sequence reads aligned to any copy of the KIV-2 domain of the LPA gene in the reference genome sequence from the number or the normalized number of the sequence reads aligned any copy of the KIV-2 domain of the LPA gene in the reference genome sequence using a GC content of the region of the LPA gene in the reference genome sequence comprising the one or more copies of the KIV-2 domain.

In some embodiments, determining the total copy number of the KIV-2 domain of the LPA gene of the subject comprises: scaling the number of copies of the region of the LPA gene comprising the one or more copies of the KIV-2 domain by a scaling factor to determine the total copy number of the KIV-2 domain of the LPA gene of the subject. The scaling factor can be based on the number of the copies of the KIV-2 domain of the LPA gene in the reference genome sequence. In some embodiments, the scaling factor is the number of the copies of the KIV-2 domain of the LPA gene in the reference genome sequence adjusted (e.g., multiplied) by a correction factor (e.g., about 1.01 to about 1.1). The correction factor can correct for sequencing bias. In some embodiments, the scaling factor is the number of the copies of the KIV-2 domain of the LPA gene in the reference genome sequence. In some embodiments, the number of copies of the KIV-2 domain of the LPA gene in the reference genome sequence is six.

In some embodiments, the method further comprises: creating a file or a report and/or generating a user interface (UI) comprising a UI element representing or comprising (i) the total copy number of the KIV-2 domain of the LPA gene of the subject and/or (iia) a number of copies of the KIV-2 domain of the LPA gene of a first allele of the subject and (iib) a number of copies of the KIV-2 domain of the LPA gene of a second allele of the subject.

In some embodiments, the method further comprises: determining a likely concentration of Lipoprotein(a) in the subject using the total copy number of the KIV-2 domain of the LPA gene of the subject. In some embodiments, the method further comprises: determining a likelihood of myocardial infarction and/or coronary arterial disease in the subject using the total copy number of the KIV-2 domain of the LPA gene of the subject and/or the likely concentration of Lipoprotein(a) in the subject.

In some embodiments, the plurality of sequence reads comprises sequence reads that are about 100 base pairs to about 1000 base pairs in length each. In some embodiments, the plurality of sequence reads comprises paired-end sequence reads and/or single-end sequence reads. In some embodiments, the plurality of sequence reads is generated by whole genome sequencing (WGS), such as clinical WGS (cWGS). In some embodiments, the sample comprises cells, cell-free DNA, cell-free fetal DNA, amniotic fluid, a blood sample, a biopsy sample, or a combination thereof.

Disclosed herein include embodiments of a system for determining a copy number of kringle IV type 2 (KIV-2) domain of LPA gene. In some embodiments, a system for determining a copy number of KIV-2 domain of LPA gene comprises: non-transitory memory configured to store executable instructions. The non-transitory memory can be configured to store a plurality of sequence reads generated from a sample obtained from a subject. The system can comprise: a hardware processor in communication with the non-transitory memory. The hardware processor can be programmed by the executable instructions to perform: aligning the plurality of sequence reads to a reference sequence (such as a reference genome sequence), comprising one or more copies of the KIV-2 domain of the LPA gene, to obtain a plurality of aligned sequence reads comprising sequence reads aligned to any copy of the KIV-2 domain of the LPA gene in the reference sequence. The hardware processor can be programmed by the executable instructions to perform: determining a number of the sequence reads aligned to any copy of the KIV-2 domain of the LPA gene in the reference sequence. The hardware processor can be programmed by the executable instructions to perform: determining a normalized, GC-corrected number of the sequence reads aligned to any copy of the KIV-2 domain of the LPA gene in the reference sequence. The hardware processor can be programmed by the executable instructions to perform: determining a number of copies of a region of the LPA gene comprising the one or more copies of the KIV-2 domain using the normalized, GC-corrected number of the sequence reads aligned to any copy of the KIV-2 domain of the LPA gene in the reference sequence. The hardware processor can be programmed by the executable instructions to perform: determining a total copy number of the KIV-2 domain of the LPA gene of the subject using (a) the number of copies of the region of the LPA gene comprising the one or more copies of the KIV-2 domain and (b) a number of copies of the KIV-2 domain of the LPA gene in the reference sequence.

In some embodiments, the hardware processor is further programmed by the executable instructions to perform: determining (a) a number of copies of the KIV-2 domain of the LPA gene of a first allele of the subject and (b) a number of copies of the KIV-2 domain of the LPA gene of a second allele of the subject, based on one or more single nucleotide variants (SNVs) of the KIV-2 domain of the LPA gene. In some embodiments, the one or more SNVs comprise T>G at position 296 and C>G at position 1264 of a copy of the KIV-2 domain of the LPA gene in the reference genome sequence. The copy of the KIV-2 domain can comprise a sequence of SEQ ID NO: 1. In some embodiments, the one or more SNVs comprise T>G at chr6:160630428, 160635977, 160641520, 160624884, 160619338, and/or 160613786 of hg38 and/or C>G at chr6:160620306, 160625852, 160631396, 160636945, 160642488, and/or 160614754 of hg38 or at corresponding positions of another reference genome sequence.

In some embodiments, a sequence read of the sequence reads aligned to any copy of the KIV-2 domain of the LPA gene in the reference genome sequence with a low alignment quality score.

In some embodiments, determining the normalized, GC-corrected number of the sequence reads aligned to any copy of the KIV-2 domain of the LPA gene in the reference genome sequence comprises: determining the normalized number of the sequence reads aligned to any copy of the KIV-2 domain of the LPA gene in the reference genome sequence using (1a) a depth of the sequence reads aligned to any copy of the KIV-2 domain of the LPA gene in the reference genome sequence, (1b) a length of the region of the LPA gene in the reference genome sequence comprising the one or more copies of the KIV-2 domain, (2a) a depth of sequence reads of the plurality of sequence reads aligned to each of a plurality of regions of the reference genome sequence other than a genetic locus comprising LPA gene, and (2b) a length of each of the plurality of regions of the reference genome other than the genetic locus comprising LPA gene. In some embodiments, determining the normalized, GC-corrected number of the sequence reads aligned to any copy of the KIV-2 domain of the LPA gene in the reference genome sequence comprises: determining the normalized, GC-corrected number of the sequence reads aligned to any copy of the KIV-2 domain of the LPA gene in the reference genome sequence from the the normalized number of the sequence reads aligned any copy of the KIV-2 domain of the LPA gene in the reference genome sequence using a GC content of the region of the LPA gene in the reference genome sequence comprising the one or more copies of the KIV-2 domain.

In some embodiments, determining the total copy number of the KIV-2 domain of the LPA gene of the subject comprises: scaling the number of copies of the region of the LPA gene comprising the one or more copies of the KIV-2 domain by the number of the copies of the KIV-2 domain of the LPA gene in the reference genome sequence to determine the total copy number of the KIV-2 domain of the LPA gene of the subject. In some embodiments, the number of copies of the KIV-2 domain of the LPA gene in the reference genome sequence is six.

In some embodiments, wherein the hardware processor is further programmed by the executable instructions to perform: creating a file or a report and/or generating a user interface (UI) comprising a UI element representing or comprising (i) the total copy number of the KIV-2 domain of the LPA gene of the subject and/or (iia) a number of copies of the KIV-2 domain of the LPA gene of a first allele of the subject and (iib) a number of copies of the KIV-2 domain of the LPA gene of a second allele of the subject.

In some embodiments, the hardware processor is further programmed by the executable instructions to perform: determining a likely concentration of Lipoprotein(a) in the subject using the total copy number of the KIV-2 domain of the LPA gene of the subject. In some embodiments, the hardware processor is further programmed by the executable instructions to perform: determining a likelihood of myocardial infarction and/or coronary arterial disease in the subject using the total copy number of the KIV-2 domain of the LPA gene of the subject and/or the likely concentration of Lipoprotein(a) in the subject.

In some embodiments, the plurality of sequence reads comprises sequence reads that are about 100 base pairs to about 1000 base pairs in length each. In some embodiments, the plurality of sequence reads comprises paired-end sequence reads and/or single-end sequence reads. In some embodiments, the plurality of sequence reads is generated by whole genome sequencing (WGS), such as clinical WGS (cWGS). In some embodiments, the sample comprises cells, cell-free DNA, cell-free fetal DNA, amniotic fluid, a blood sample, a biopsy sample, or a combination thereof.

Also disclosed herein include a non-transitory computer-readable medium storing executable instructions, when executed by a system (e.g., a computing system), causes the system to perform any method or one or more steps of a method disclosed herein.

Details of one or more implementations of the subject matter described in this specification are set forth in the accompanying drawings and the description below. Other features, aspects, and advantages will become apparent from the description, the drawings, and the claims. Neither this summary nor the following detailed description purports to define or limit the scope of the inventive subject matter.

### BRIEF DESCRIPTION OF THE DRAWINGS

FIGS. 1A-1B show the complex repeat structure around KIV-2 of the LPA gene. One copy of each of KIV-2 domains 1-10 is shown in FIG. 1A. A human reference genome sequence, such as GRCh38, can include six copies of KIV-2 as illustrated in FIG. 1B.

FIG. 2 depicts an exemplary alignment of short Illumina reads and long Pacific Biosciences (PacBio) HiFi and Oxford Nanopore Technologies (ONT) reads to the LPA gene.

FIG. 3A-FIG. 3B are non-limiting exemplary plots comparing KIV-2 allele lengths against PacBio and Bionano validations. Shown are comparisons of KIV-2 copy number as determined by kiv2CN or in validations from Bionano optical mapping or PacBio HiFi reads. FIG. 3A depicts a non-limiting exemplary plot showing comparisons of allele lengths where the per-allele copy number was available from kiv2CN and one of the validation technologies. FIG. 3B depicts a non-limiting exemplary plot showing that as kiv2CN can always report total copy number, this was also compared in cases where Bionano or PacBio successfully reported copy number for both alleles. Dashed lines indicate error margins of 5% from expected.

FIG. 4A-FIG. 4B are non-limiting exemplary plots comparing KIV-2 allele lengths in 1 kG parents vs. offspring as measured by the kiv2CN tool. FIG. 4A is a non-limiting exemplary plot showing that for 60 trios where both offspring and parent KIV-2 allele lengths are reported, an allele combination was chosen which minimizes the total difference between each of the two offspring alleles and one from each parent as the most likely allele origin. Each pair, consisting of one offspring allele and one associated parent allele, is shown for a total of 120 allele pairs. Dashed lines indicate error margins of 5% from expected. FIG. 4B is non-limiting exemplary plot showing that for 153 duos where both offspring KIV-2 allele lengths and those from one parent are reported, an allele combination was chosen which minimizes the difference between one of the two offspring alleles and one from the parent where both are known. Each pair, consisting of one offspring allele and one associated parent allele, is shown for a total of 153 allele pairs. Dashed lines indicate error margins of 5% from expected.

FIG. 5 shows histograms of exemplary distributions of KIV-2 repeats among different ethnic samples of 1000 g datasets. Africa, AFR; Admixed American, AMR; European, EUR; East Asian, EAS; South Asian, SAS.

FIG. 6 shows histograms of exemplary distributions of the phased copy number variant (CNV) differences among various ethnicities of 1000 g datasets. Each difference shown is the haplotype difference between allele 1 CN and allele 2 CN of KIV-2 of a subject.

FIG. 7 shows histograms of exemplary distributions of KIV-2 repeats among various ethnicities.

FIG. 8 shows histograms of exemplary distributions of the phased copy number variant (CNV) differences among various ethnicities. Each difference shown is the haplotype difference between allele 1 CN and allele 2 CN of KIV-2 of a subject.

FIGS. 9A-9B illustrate determining the total copy number of the KIV-2 domain of a subject and phasing (or determining) the copy numbers of the KIV-2 domain of the two alleles of the subject.

FIG. 10 shows a flowchart of an exemplary method described herein.

FIG. 11 shows exemplary coverage plots of 18 haplotype assemblies across the KIV-2 locus. Gaps in coverage indicate regions where assemblies failed to span the KIV-2 repeat, leaving full allelic length unknown.

FIG. 12 shows histograms of exemplary KIV-2 CNV distributions among EUR subgroups (179 Utah Residents with European Ancestry, CEU; 99 Finnish, FIN; 91 British, GBR; 157 Iberian, IBS; and 107 Toscani, TSI).

FIG. 13 shows histograms of exemplary KIV-2 CNV haplotype difference distribution among EUR subgroups (303 samples total). Each difference shown is the haplotype difference between allele 1 CN and allele 2 CN of KIV-2 of a subject.

FIG. 14 shows a non-limiting exemplary Empirical Cumulative Distribution Function plot for CNV estimate distribution of all ethnicities in the 1000 g dataset.

FIG. 15 shows a non-limiting exemplary Empirical Cumulative Distribution Function plot for phased CNV estimate difference i.e. abs (allele1_CN-allele2_CN) distribution of all ethnicities in 1000 g dataset.

FIG. 16 shows histograms of KIV-2 CNV distributions among AFR subgroups (178 Yoruba, YRI; 99 Mende, MSL; 99 Luhya, LWK; 178 Gambian Mandinka, GWD; 149 Esan, ESN; 74 African Ancestry South-West United States, ASW; 116 African Caribbean, ACB).

FIG. 17 shows histograms of KIV-2 CNV haplotype difference distribution among AFR subgroups (total 462 samples). Each difference shown is the haplotype difference between allele 1 CN and allele 2 CN of KIV-2 of a subject.

FIG. 18 shows a non-limiting exemplary Empirical Cumulative Distribution Function plot for CNV estimate distribution of all ethnicities in Atherosclerosis Risk in Communities (ARIC) dataset.

FIG. 19 shows a non-limiting exemplary Empirical Cumulative Distribution Function plot for phased CNV estimate difference i.e. abs (allele 1 CN-allele 2 CN) distribution of all ethnicities in ARIC dataset.

FIG. 20 is a flow diagram showing an exemplary method of determining a copy number of kringle IV type 2 (KIV-2) domain of LPA gene.

FIG. 21 is a block diagram of an illustrative computing system configured to determine a copy number of kringle IV type 2 (KIV-2) domain of LPA gene.

Throughout the drawings, reference numbers may be re-used to indicate correspondence between referenced elements. The drawings are provided to illustrate example embodiments described herein and are not intended to limit the scope of the disclosure.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Understanding the genomic complexity of adult diseases such as cardiovascular disease (CVD) is the next frontier in genomics. This requires unprecedented accuracy and scaling to identify common mechanisms to decipher diseases that emerge only after ˜30+ years of living. To approach this, all types of variations of a human genome are needed including SNV (single nucleotide variant) and SV (structural variant)/CNV (copy number variant) as well as coding and non-coding variations with novel analytical methodologies. SV and CNV are the largest source of genetic diversity and have shown to impact human diseases. Cardiovascular disease is one of the deadliest diseases in the modern world. One person dies every 36 seconds in the United States from cardiovascular disease. Much of a person's risk of CVD is genetically predetermined, but can be circumvented with proper treatment and lifestyle changes. Still, the detection and characterization remains challenging.

One of the clearest relations of gene to protein to disease for coronary heart disease (CHD) is Lipoprotein(a) (Lp(a)). Lp(a) shows an extremely high heritability (˜70 to >90%) across European, Asian and African populations. Thus making understand the impact and structure of the Lp(a) gene (LPA) a clear target for study. LPA evolved from plasminogen (PLG) very recently, which is characterized by over five different paralogous kringle domains (kringles I-V (KI-KV)). Given multiple expansions and deletions, the human lineage has around 10 kringle domains. One of the most impactful for LPA is KIV-2 that is repeated in tandem between 5 to 50+ copies. KIV-2 is a 5.5 kbp large repeat that includes two exons. Thus, the number of KIV-2 repeats directly impacts the length of the mRNA, which consists of ˜70% of the two exons. The length of LPA is inversely correlated to the amount of Lp(a) protein and to the risk of CHD. Most notable is that the copy number of KIV-2 is predetermined by birth and is not reported to change over the lifetime. Nevertheless, large variation of Lp(a) levels exists between individuals but also between different human populations and non-human primates. As an example, on average, African populations have 2 to 3 fold higher Lp(a) concentrations than Europeans or Asian populations.

Given the complexity of the KIV-2 repeat, it is often impossible to determine the number of copies with traditional sequencing alone. Therefore, several marker SNVs have been suggested that are commonly outside of KIV-2 repeat but within the LPA gene. These marker SNVs are in strong linkage with certain CNV numbers of KIV-2 repeats. For example, rs10455872+rs3798220 are often used marker SNVs that work well in Europeans. Thus, they are even used for commercial kits to determine CHD risk (42 and 57% respectively for both SNVs). However, these SNVs have shown no association in Japan (only other ethnic studies) or Hispanics. The SNVs are generally absent in autochthonous Africans, low frequency in African-Americans and Europeans, but are in high frequency in Asian and South and Central Americans (Mexicans, Columbians, Puerto Ricans, Peruvians). Thus, like other marker SNVs, they are only in strong linkage disequilibrium (LD) within a certain population but proven to not be causative and thus not reliable. This makes the precise determination of the number of KIV-2 repeats a necessity for regular genetic sequencing-based essays. This remains challenging even for longer read essays to capture and phase both copies of KIV-2 repeat units.

Disclosed herein is a short-read CN caller that determines the total number of copies of the KIV-2 repeat. The caller is referred to herein as kiv2CN. kiv2CN can implement a method of determining a copy number of KIV-2 domain or repeat disclosed herein. Kiv2CN can report the KIV-2 copy numbers determined.

Disclosed herein include methods of determining a copy number of kringle IV type 2 (KIV-2) domain (or repeat) of LPA gene. In some embodiments, a method of determining a copy number of KIV-2 domain of LPA gene is under control of a processor (e.g., a hardware processor or a virtual processor) and comprises: receiving a plurality of sequence reads generated from a sample obtained from a subject (which can be a mammal, such as a human). The method can comprise: aligning the plurality of sequence reads to a reference genome sequence, comprising one or more copies of the KIV-2 domain of the LPA gene, to obtain a plurality of aligned sequence reads comprising sequence reads aligned to any copy of the KIV-2 domain of the LPA gene in the reference genome sequence. The method can comprise: determining a number of the sequence reads aligned to any copy of the KIV-2 domain of the LPA gene in the reference genome sequence. The method can comprise: determining a number of copies of a region of the LPA gene comprising the one or more copies of the KIV-2 domain based on the number of the sequence reads aligned to any copy of the KIV-2 domain of the LPA gene in the reference genome sequence. The method can comprise: determining a total copy number of the KIV-2 domain of the LPA gene of the subject using (a) the number of copies of the region of the LPA gene comprising the one or more copies of the KIV-2 domain and (b) a number of copies of the KIV-2 domain of the LPA gene in the reference genome sequence.

Disclosed herein include embodiments of a system for determining a copy number of kringle IV type 2 (KIV-2) domain of LPA gene. In some embodiments, a system for determining a copy number of KIV-2 domain of LPA gene comprises: non-transitory memory configured to store executable instructions. The non-transitory memory can be configured to store a plurality of sequence reads generated from a sample obtained from a subject. The system can comprise: a hardware processor in communication with the non-transitory memory. The hardware processor can be programmed by the executable instructions to perform: aligning the plurality of sequence reads to a reference sequence (such as a reference genome sequence), comprising one or more copies of the KIV-2 domain of the LPA gene, to obtain a plurality of aligned sequence reads comprising sequence reads aligned to any copy of the KIV-2 domain of the LPA gene in the reference sequence. The hardware processor can be programmed by the executable instructions to perform: determining a number of the sequence reads aligned to any copy of the KIV-2 domain of the LPA gene in the reference sequence. The hardware processor can be programmed by the executable instructions to perform: determining a normalized, GC-corrected number of the sequence reads aligned to any copy of the KIV-2 domain of the LPA gene in the reference sequence. The hardware processor can be programmed by the executable instructions to perform: determining a number of copies of a region of the LPA gene comprising the one or more copies of the KIV-2 domain using the normalized, GC-corrected number of the sequence reads aligned to any copy of the KIV-2 domain of the LPA gene in the reference sequence. The hardware processor can be programmed by the executable instructions to perform: determining a total copy number of the KIV-2 domain of the LPA gene of the subject using (a) the number of copies of the region of the LPA gene comprising the one or more copies of the KIV-2 domain and (b) a number of copies of the KIV-2 domain of the LPA gene in the reference sequence.

Identification of Allele-Specific HIV-2 Repeats Among Multi-Ethnic Groups and Association with Lp(a) Measurements

Studies on the human LPA gene have found evidence that the kringle IV type 2 (KIV-2) variable number tandem repeats (VNTR) is one of the controlling factors of lipoprotein(a) (Lp(a)) isoform size. The LPA gene, including the KIV-2 variant, determines the Lp(a) protein level (a high number of KIV-2 repeats is associated with low Lp(a) concentration) and has strong associations with cardiovascular diseases. Nevertheless, it remains challenging to determine the number of KIV-2 repeats in whole-genome sequencing (WGS) data due to the repetitiveness of KIV-2. Lp(a) is currently widely studied among Europeans, and studies have revealed clear associations with cardiovascular risk. However, it remains challenging to extend these insights to other ethnicities including Hispanics due to the lack of genetic and phenotypic data available on non-Europeans. Thus, an allele-specific copy number (CN) estimation of KIV-2 is needed to improve the genetic diagnosis and understanding of the impact of KIV-2 on cardiovascular risk across ethnicities.

Using data from different cohort studies, the association of KIV-2 repeats with Lp(a) concentrations and cardiovascular risk prediction was studied. To achieve this, a novel approach was developed to directly assess KIV-2 levels derived from Illumina WGS datasets. This method was carefully benchmarked against Pacbio HiFi based assemblies to ensure high accuracy and precision. A WGS dataset of randomly selected 3,020 participants (samples sequenced on Illumina HiSeq X and mapped to GRCh38 reference sequence) from multiple ethnic groups including 1000 European samples, 1019 African-American samples from the Atherosclerosis Risk in Communities (ARIC) cohort study, and 1001 Hispanic samples from the Hispanic Community Health Study and the Study of Latinos (HCHS/SOL).

The tool (kiv2CN) estimated the summed copy number of both alleles in all samples and performed haplotype phasing of ˜46% of the samples (45.9% Europeans, 51.3% African-Americans, and 40.5% Hispanics). The frequency distribution of CN estimates among three ethnic groups showed that the African-American group has a higher percentage (˜70%) of samples that are in KIV-2 repeats ranging from 20 to 40 versus ˜45% for the Hispanic group. Using these KIV-2 CN estimates, the results of an association study, which utilized protein measurements and health records from each of these individuals, are described below. Differences in KIV-2 CN that were identified across the different ethnicities are presented below. The methods described herein can enable improved diagnosis of cardiovascular disease risks among understudied ethnicities.

### Results

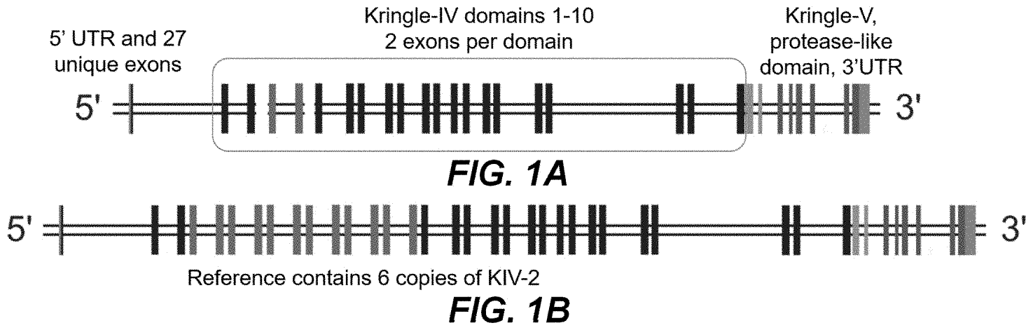

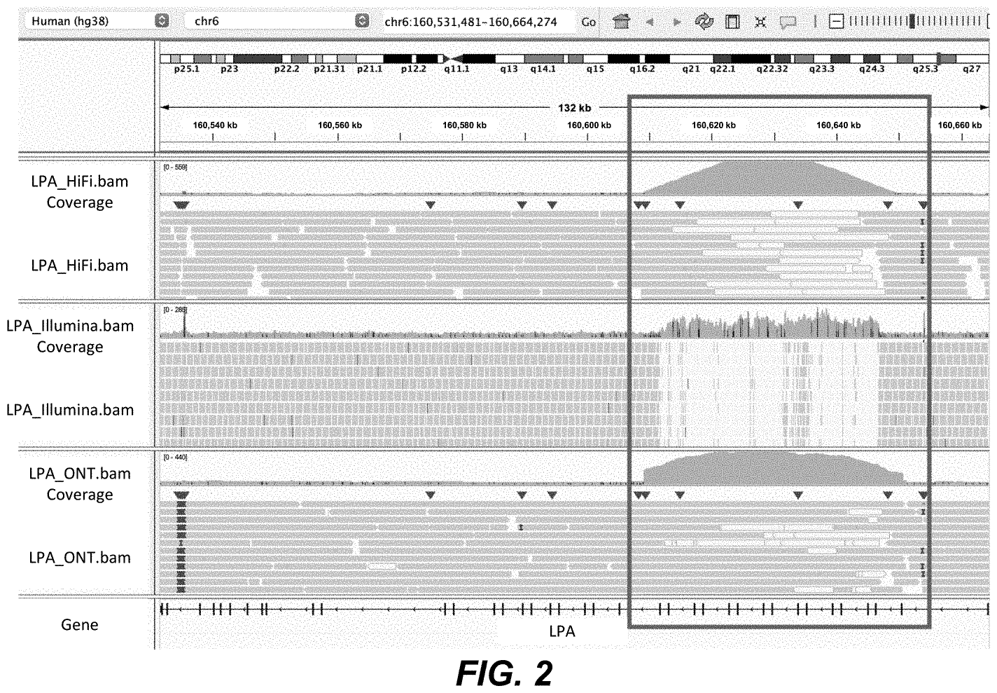

The investigation of the gene LPA remains challenging given its complex repeat structure around KIV-2 (See FIG. 1). This was observed when reads were aligned to the GRCh38 version of the human reference genome. FIG. 2 shows many white reads in the region of KIV-2, which are indicative of mapping quality zero. The GRCh38 representation of KIV-2 contains six copies of the 5.5 kbp repeat (See FIG. 1). Even for longer reads (Pacbio HiFi), this region is challenging, indicated by many MQ=0 mapping reads and the same was observed for Oxford Nanopore Technologies (ONT) (FIG. 2). Depending on the overall read length, the ratio of non-unique mapped reads increases, which has often hindered the direct assessment of KIV-2 repeat levels.

kiv2CN—WGS-Based Copy Number Caller of the KIV-2 Repeat in LPA

To overcome the foregoing and assess this critical information for cardiovascular disease (CVD), kiv2CN was implemented. kiv2CN estimates KIV-2 copy number (CN) by counting reads that align to any of the 6 KIV-2 repeat copies in the reference genome, including reads aligned with a mapping quality of zero. The summed read count was normalized and corrected for GC content to derive the KIV-2 CN.

The calculated KIV-2 CN is the sum of two alleles. kiv2CN calls allele-specific CNs in a subset of samples. Two common intronic single nucleotide polymorphisms (SNPs) were observed in the KIV-2 repeat region (T>G at chr6:160630428/160635977/160641520/160624884/160619338/160613786 and C>G at chr6:160620306/160625852/160631396/160636945/160642488/160614754, hg38). When these two SNPs are found on an allele, all copies of the KIV-2 repeat on the same allele carry these two SNPs. kiv2CN uses the ratio of supporting reads at these two SNPs to calculate allele-specific CNs in samples where one allele carries the SNPs and the other allele doesn't.

The accuracy of kiv2CN was validated against different long read technologies. First, Pacbio HiFi sequence data was used to de novo assemble the KIV-2 regions of five different samples: NA12878, NA24631 (HG005), NA24385 (HG002), NA19238 and NA19239 (shown in Table 1). Additionally, for the HG002 dataset, the two KIV-2 haplotypes were able to be phased using the disclosed methods.

HG001/NA12878 was picked as the control sample for estimating copy number variant (CNV) using Illumina short reads. Though it is extremely challenging to compute CNVs using short reads, kiv2CN has done remarkably well, with a CNV estimation value 37.6 which is very close to the CNV estimation value 38 that is computed by using Pacbio HiFi reads. Next, HG002, another control sample that is mostly used for various genomic analyses, was used to estimate CNV using Illumina short reads. For this control sample, the haplotypes were surprisingly able to be phased. The comparison of CNV values estimated by kiv2CN with the HiFi based assembly method (37.6 and 38 respectively) confirms that the disclosed methods perform extremely well for the 2nd control sample as well. The phased CNV estimates (13.2 and 24.5) by kiv2CN are also very close to the phased CNV estimates (14 and 24) of HiFi assembly-based methods.

With the use of three other samples, it was confirmed that the CNV estimation of disclosed methods is comparable to PacBio HiFi based assembly method for all samples with differences ranging from 0.4 to 1.8.

TABLE 1
Comparison of CNV estimates with Illumina-based CN
calls determined using kiv2CN and Pacbio HiFi based assembly

| Sample | Illumina | PacBio HiFi assembly |
|---|---|---|
| NA12878 | 37.6 | 38 |
| HG005 (NA24631) | 43.2 | 45 |
| HG002 (NA24385) | 37.6 (13.2/24.5) | 38 (14/24) |
| NA19238 | 35.1 | 36 |
| NA19239 | 22.7 | 21 |

Platinum Genome Pedigree. In the Platinum Genome pedigree, while kiv2CN was not able to phase the two alleles due to the absence of the differentiating SNPs, the children were expected to have 4 different pairs of haplotypes and the children with the same haplotype combinations were found to have almost identical CN calls (Table 2).

TABLE 2
Children with the same genotype have
very close Illumina-based CN calls

| | | | CN |
|---|---|---|---|
| | Sample | Haplotype | Illumina |
| Parents | NA12878 | CD | 37.6 |
| | NA12877 | AB | 23.6 |
| Children | NA12888 | CA | 31.7 |
| | NA12882 | CA | 31 |
| | NA12893 | DB | 30.8 |
| | NA12887 | DB | 31.5 |
| | NA12884 | DB | 31.8 |
| | NA12879 | DB | 30 |
| | NA12886 | DA | 27 |
| | NA12881 | DA | 26.3 |
| | NA12883 | DA | 26.5 |
| | NA12880 | CB | 36.9 |
| | NA12885 | CB | 35.6 |

Comparisons. KIV-2 copy number calls from kiv2CN against calls from 53 genomes mapped was compared with Bionano optical mapping, publicly released by the Human Genome Structural Variant Consortium and by the Human Pangenome Reference Consortium (HPRC). kiv2CN calls were also compared against 8 PacBio HiFi genomes from HPRC (See, “KIV-2 assembly with PacBio HiFi reads” below).

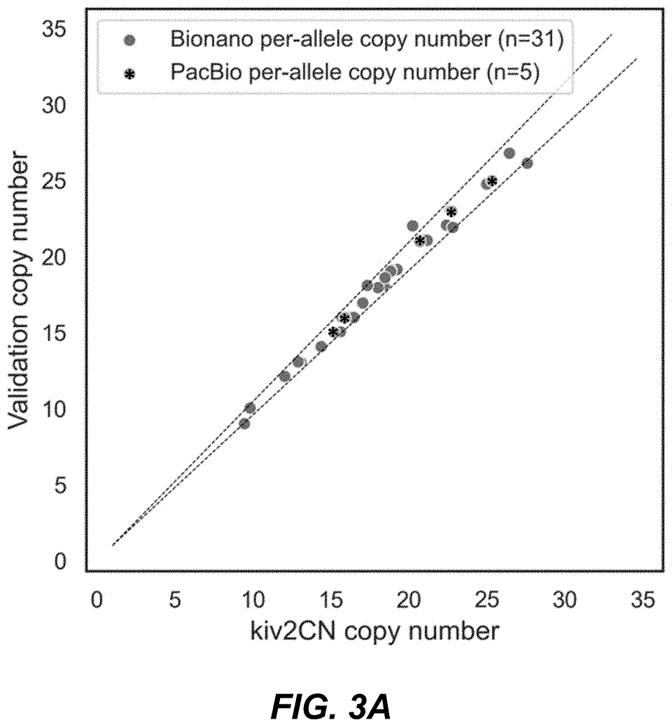

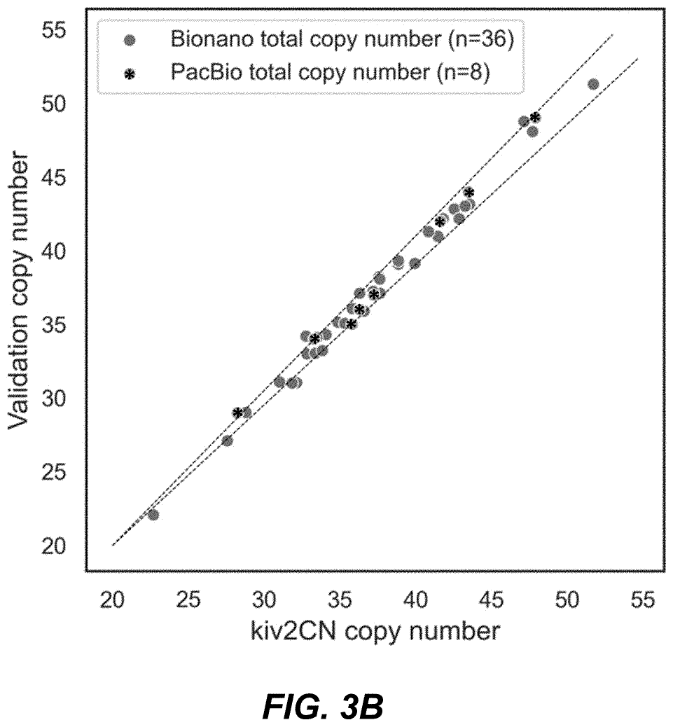

Bionano optical maps represent an orthogonal technology with high accuracy for large structural variant (SV) recall, an excellent match for validation of kiv2CN calls. Bionano mapping failed to span the full repeat locus in some cases but was successful for 87 alleles. 30 of these alleles received a kiv2CN allelic copy number call, enabling a direct comparison of copy number calls. Total copy numbers as reported by kiv2CN were also compared against the sum of allelic copy numbers from Bionano in cases where both alleles were reported by Bionano. The results of these comparisons demonstrate a high rate of concordance and showcase the accuracy of kiv2CN (FIG. 3A-FIG. 3B).

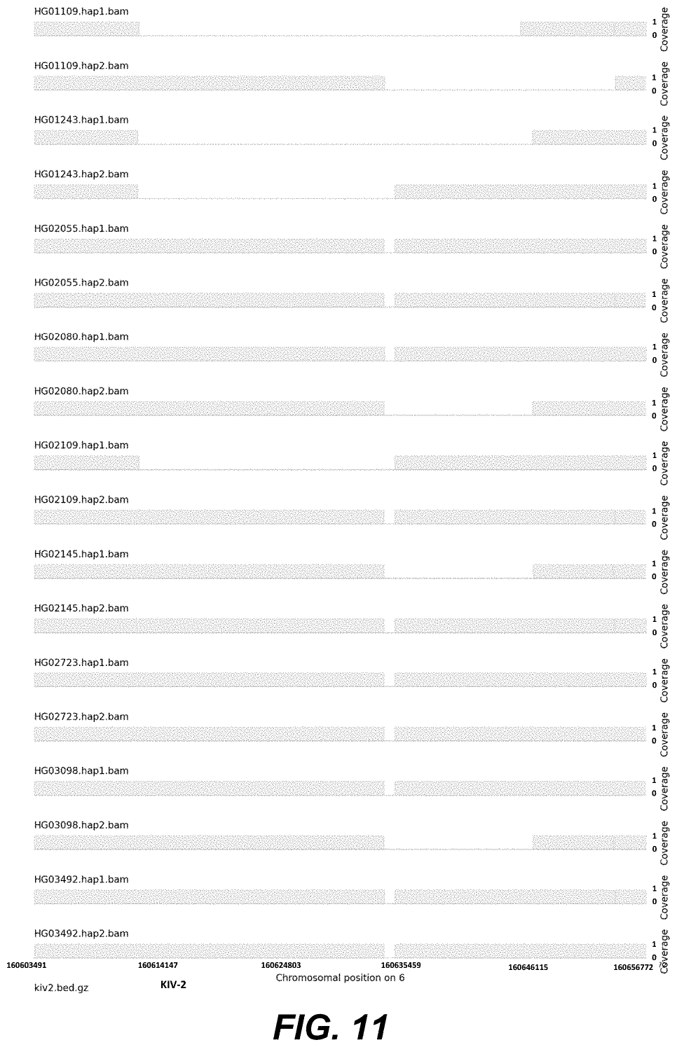

PacBio assembly of the KIV-2 repeat remains challenging due to the length and copy number of the repeat (See, “KIV-2 assembly with PacBio HiFi reads” below, FIG. 11). However, both alleles were successfully assembled for 8 samples, enabling comparison of per-allele and total copy number against kiv2CN calls (FIG. 3A-FIG. 3B). The difficulties faced in spanning the KIV-2 repeat locus with either of these long-contig technologies (e.g., Bionano) highlight the complexity of KIV-2 as well as a major strength of kiv2CN. In each sample, regardless of performance by PacBio or Bionano approaches, kiv2CN reports at least the total copy number, even if allelic copy number cannot be determined. Without being bound by any particular theory, this success is due to the kiv2CN depth-based approach, which does not lose efficacy for especially long or repetitive alleles.

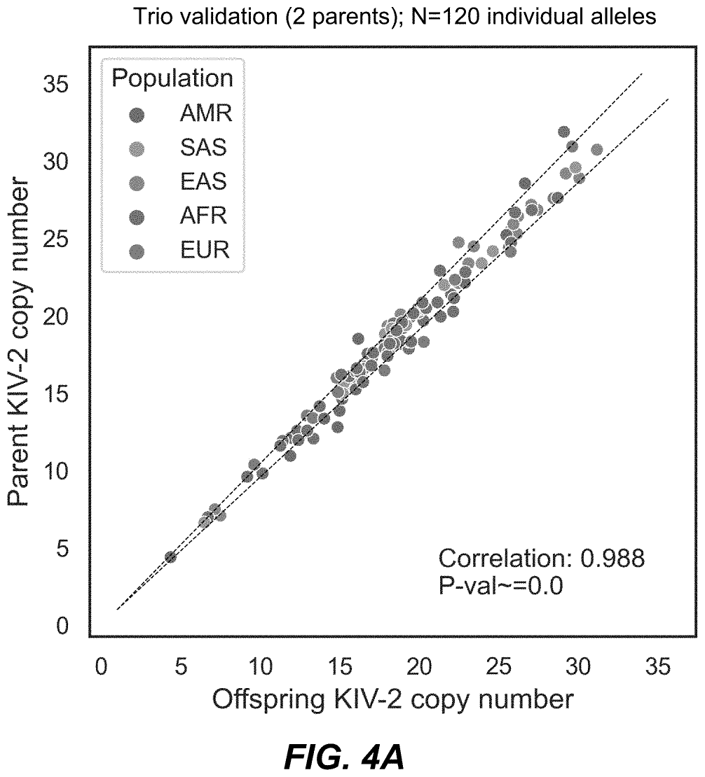

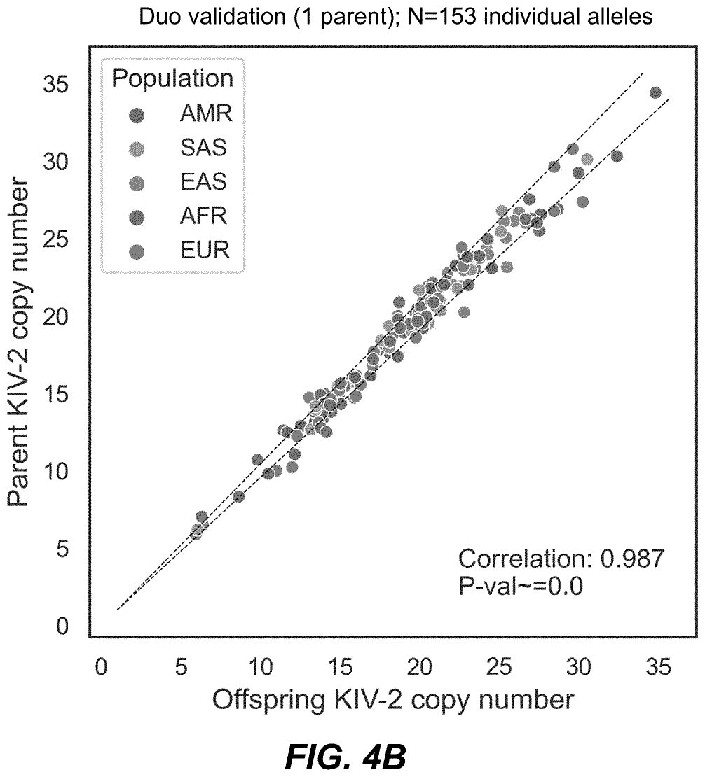

The consistency of allele-specific CN calls in 1 kGP trios was also examined. kiv2CN called allele-specific CNs in all three samples of a trio in 60 trios, so for the two alleles in the proband, the inherited parental alleles could be identified (one from each parent, based on the smallest size difference), as shown in FIG. 4A. In another 153 trios, kiv2CN called allele-specific CNs in the proband and one parent, so the inherited parental allele for one allele in the proband could be identified (the pair with the smallest size difference), as shown in FIG. 4B. The size difference between the observed allele in the proband and the inherited allele was compared. Among 120 alleles in the trio comparison, the size difference between the observed and inherited allele is within ±5% in 93 cases or 77.5% of alleles. Among 153 alleles in the duo comparison, the size difference between the observed and inherited allele is within ±5% in 115 cases or 75.2% of alleles. This translates to a CN difference of <1 given a median haplotype CN of 15.

### Diversity of KIV-2 Repeats Across Ethnicities

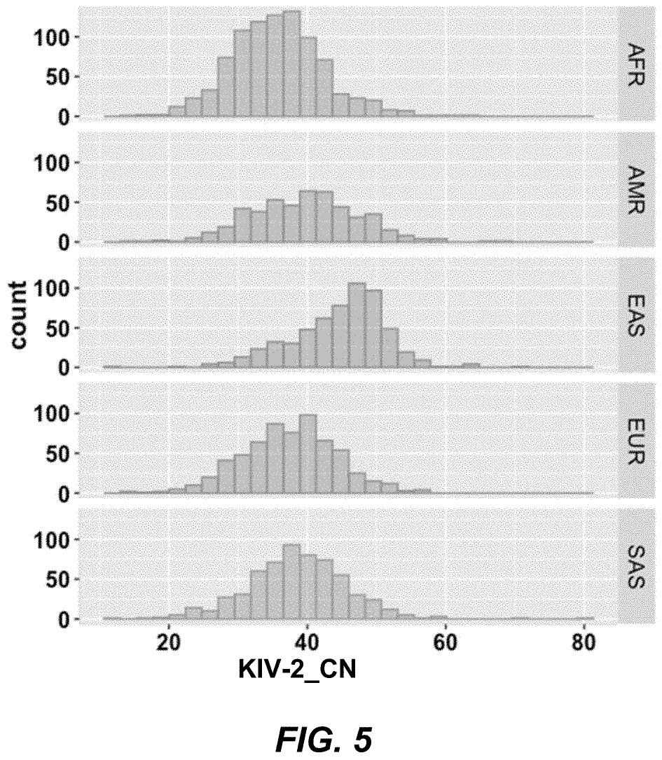

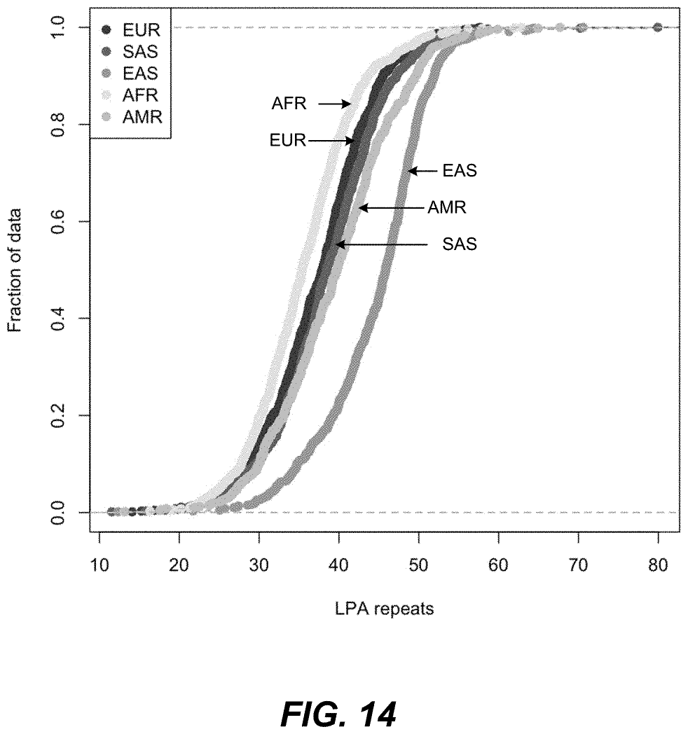

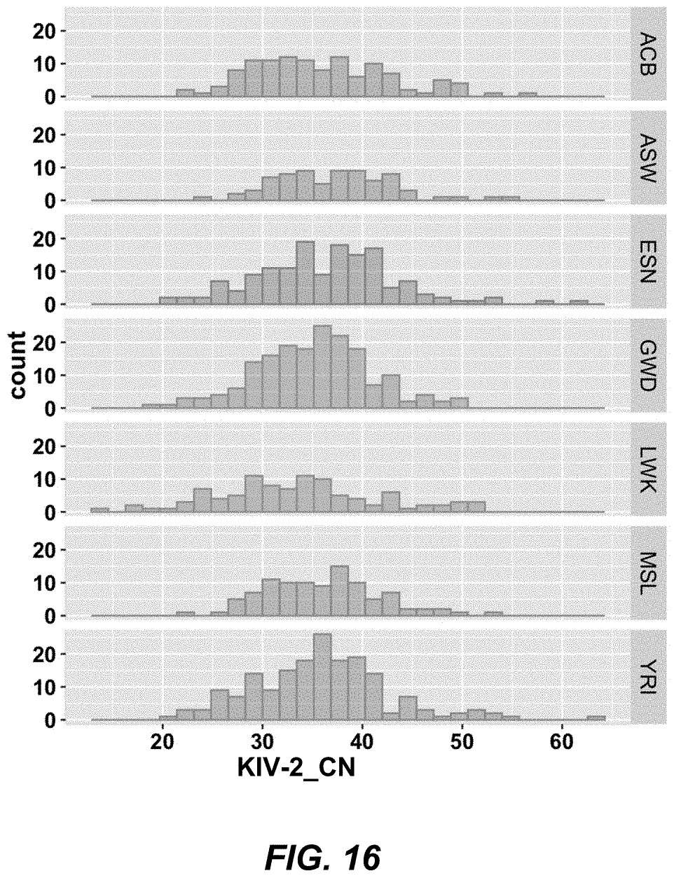

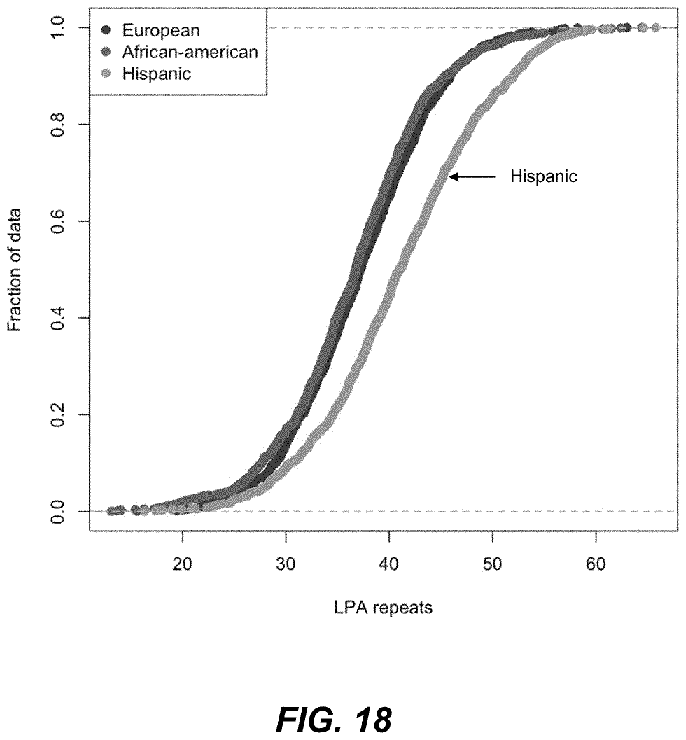

FIGS. 5, 7, 12, and 16 depict histograms illustrating the results of determining diversity of KIV-2 repeats across ethnicities. FIGS. 14 and 18 depict Empirical Cumulative Distribution Function plots illustrating the results of determining diversity of KIV-2 repeats across ethnicities.

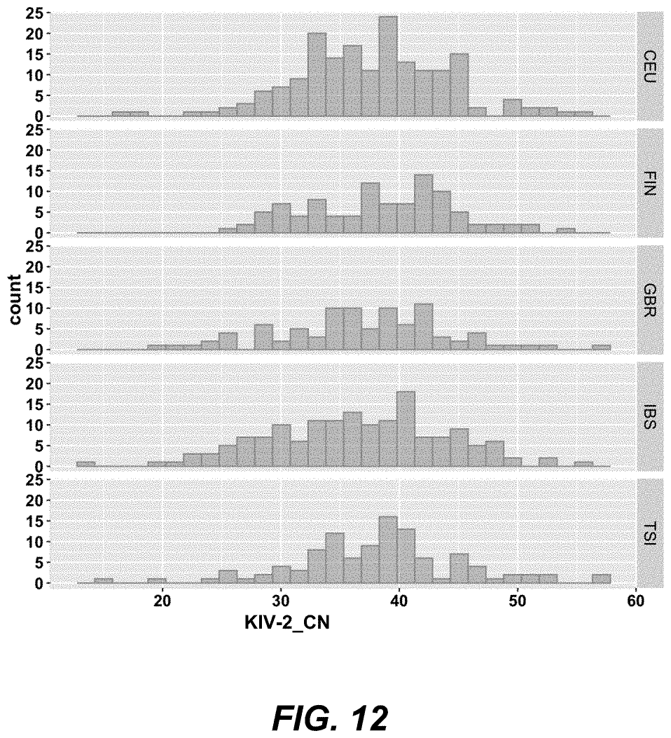

The KIV-2 CNV estimates among all 3,202 samples of 1000 genome dataset that includes 5 different ethnicities were examined: African, AFR; Admixed American, AMR; European, EUR; East Asian, EAS; and South Asian, SAS. The distribution of KIV-2 CNVs among these ethnicities are shown in FIG. 5. First, the distribution among 633 European population samples was studied which consists of 5 subgroups (Utah Residents with European Ancestry, CEU; Finnish, FIN; British, GBR; Iberian, IBS; and Toscani, TSI). The majority (˜70%) of the population have KIV-2 CNVs in the range of 30-45 with average CNV estimate 37.5 and standard deviation of 6.86. A two-sample Kolmogorov-Smirnov test was used to compare the CNV distribution of EUR samples with CNV distributions of other samples. The EUR and SAS have an almost identical distribution with p-value=0.04722. The distribution was slightly different when compared to AFR and AMR (as shown in ECDF plot, FIG. 14) with p-values 1.567e-08 and 7.336e-06 respectively. However, a big difference was observed when compared to EAS samples with a very low p-value (<2.2e-16). The distribution was further studied among five different subgroups within the European population and it was observed that the distribution looks almost similar among all these subgroups (FIG. 12).

The study on the KIV-2 repeat distribution was further expanded among other non-European ethnicities such as Africans, Americans, and Asians (south and east). For the AFR dataset, the average CNV was 35.7 with standard deviation 6.68. The distribution of CNVs among the African population was observed to be different from other ethnicities with several peaks in the range of 30 to 40. Note that there are a higher number (893) of samples for the African population in the 1000 genome dataset. The low p-values (<2.2e-16) of both AMR and EAS from Kolmogorov-Smirnov test with AFR as the reference distribution shows that the CNV distribution of the former two are different from AFR. The higher Kolmogorov-Smirnov statistic i.e. D=0.55552 of the EAS sample confirms that distribution of East Asian samples is also different from AFR as was observed in the EUR study. For AMR samples, the Kolmogorov-Smirnov statistic when compared to AFR was 0.26431. The AMR group has the lowest number of samples (490) and it was observed that the AMR (FIG. 5) population has a more flat distribution as compared to others. The average CNV estimate for AMR was 39.6 with standard deviation 7.74. The South Asian population has almost the same number of samples as the European population and both the distributions are observed to be more similar than any other pairs. The East Asian population has a very different distribution than all other ethnicities. Almost 40% of the samples have CNV in the range of 46 to 50 and the average CNV estimate and standard deviation are 44.6 and 6.77 respectively.

Haplotype Phasing

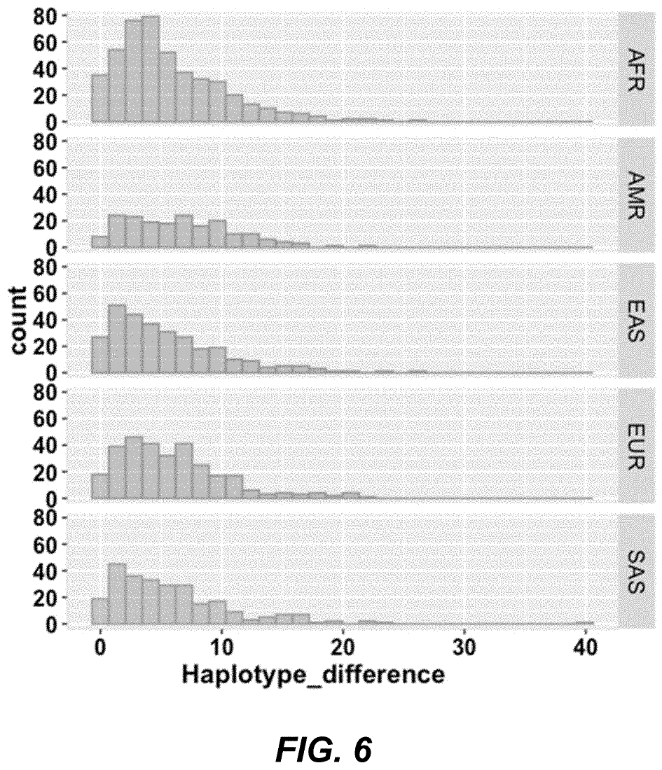

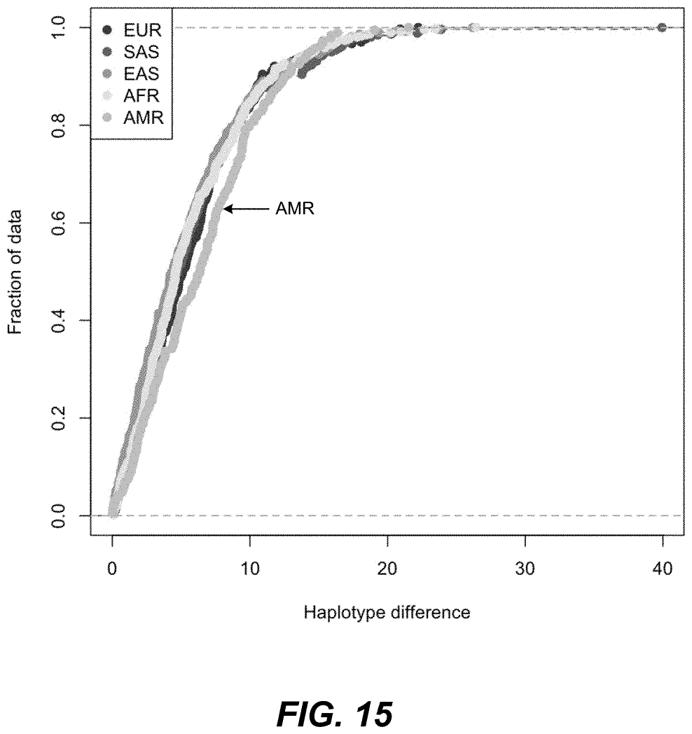

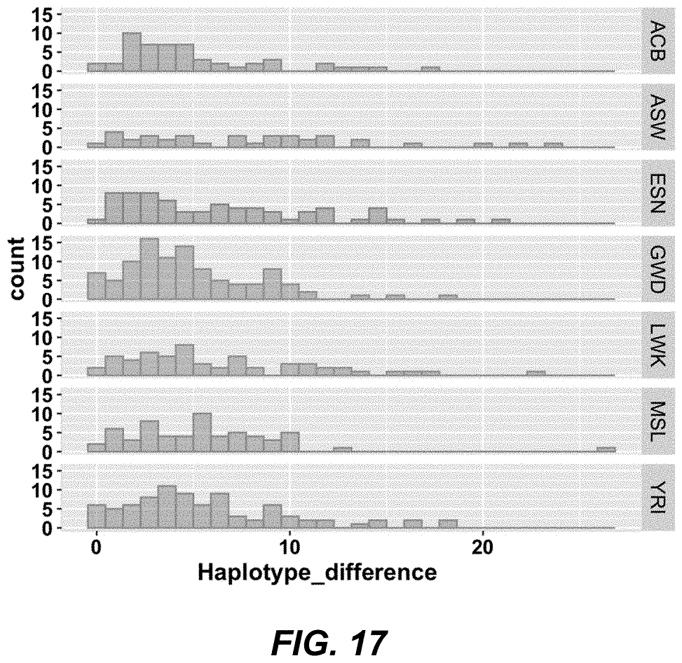

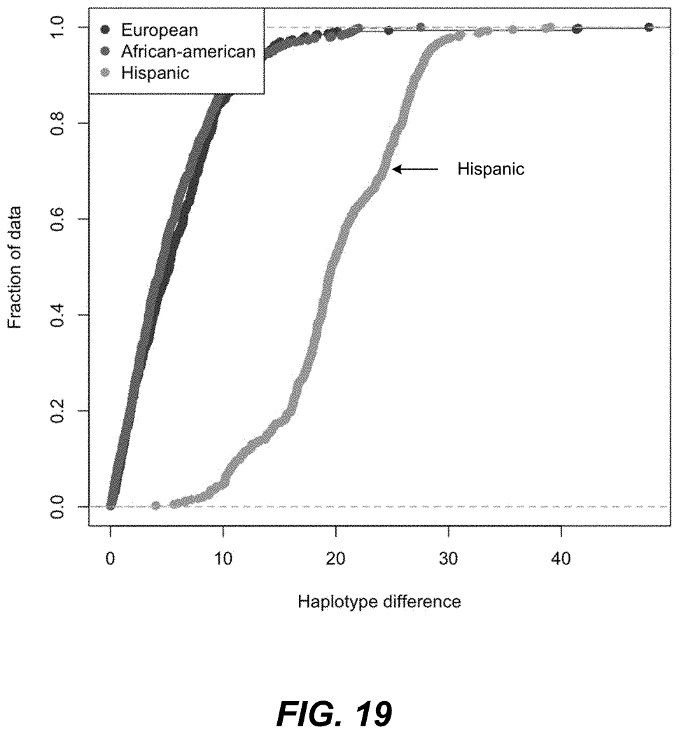

FIGS. 6, 8, 13, 17 depict histograms illustrating the results of haplotype phasing. FIGS. 15 and 19 depict Empirical Cumulative Distribution Function plots illustrating the results of haplotype phasing.

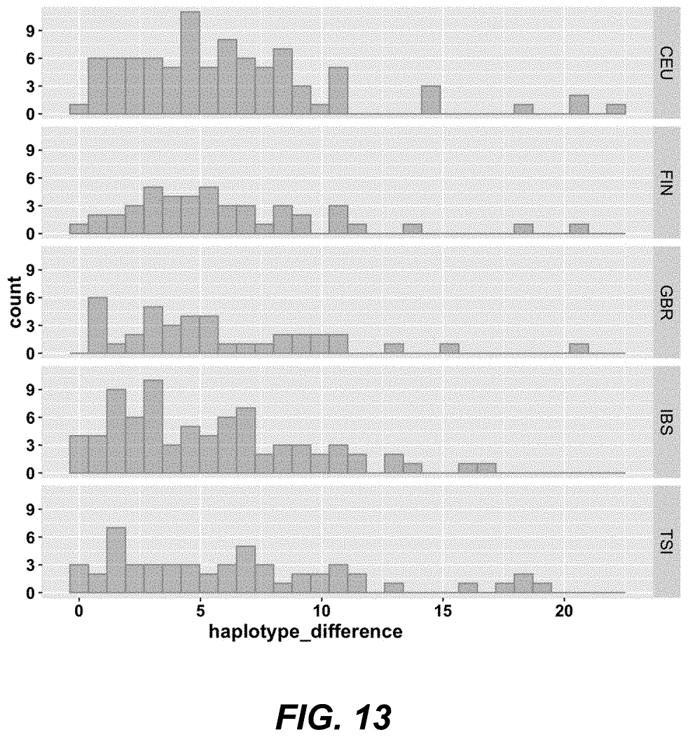

kiv2CN was able to phase the CNV estimates for ˜50% of the samples of the 1000 g dataset. The phased CNV estimates and their differences were studied for all the ethnicities. The distribution of phased CNV differences are shown in FIG. 6. For EUR samples, kiv2CN was able to estimate phased CNVs for ˜48% (303 out of 633) samples and the majority of all populations among all subgroups have the haplotype difference i.e. difference between two phased CNVs are in the range of 0 to 10 (FIG. 13). A two-sample Kolmogorov-Smirnov (KS) test was also performed by taking the distribution for EUR samples as reference to compare the distribution with other non-European groups. The high p-values with SAS and AFR (0.419 and 0443 respectively) confirms the much similar distribution as shown in FIG. 6 (also, See, ECDF plot in FIG. 15). The distribution between EUR and AMR was observed to be different (KS test p-score 0.0365) and, without being bound by any particular theory, this could be due to the low number of AMR samples that are present in the 1000 g dataset and only 186 (vs 303 EUR samples) samples have phased CNV estimates.

Association with Lp(a) Measurements

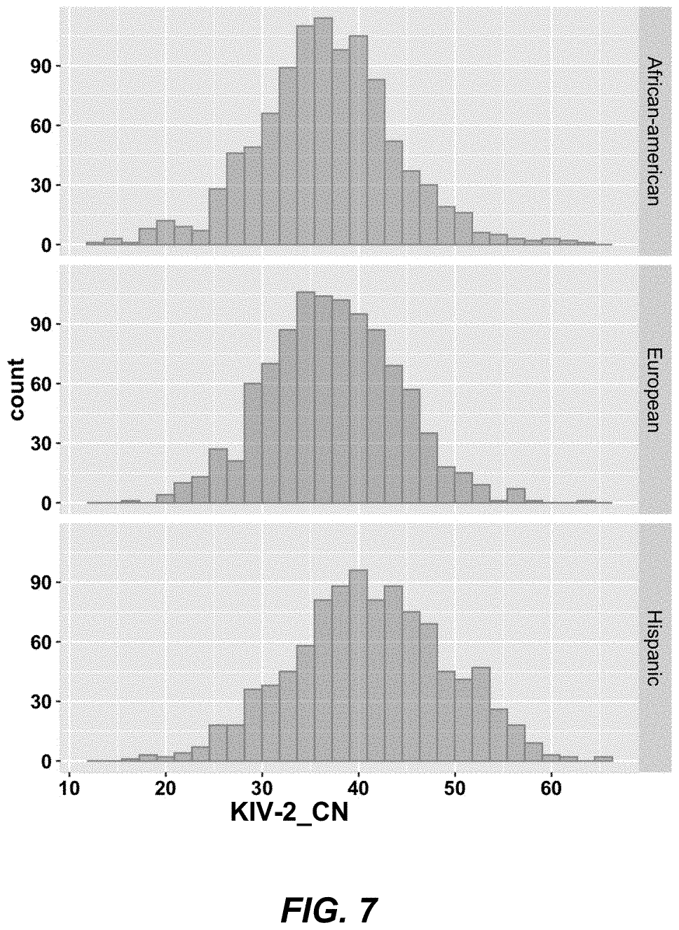

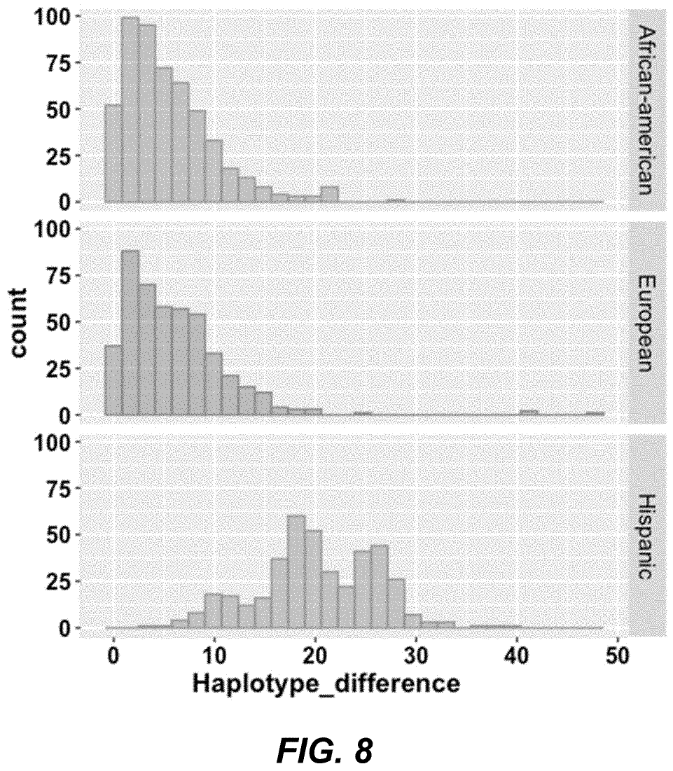

To study the association of cardiovascular risks that are related to different ethnicities, a ˜30×WGS dataset of randomly selected 3,006 participants was used: 1000 European, 1005 African-American from the Atherosclerosis Risk in Communities (ARIC) cohort and 1001 Hispanic from the Hispanic Communities Health Study (HCHS)/Study Of Latinos (SOL) cohort. These datasets are Illumina HiSeq X sequences mapped to the GRCh38 reference genome. The distribution of KIV-2 repeats among different ethnicities (FIG. 6) showed that ˜50% of the African-American population has repeats in the range of 34 to 42, while ˜50% of the Hispanic population has repeats in the range of 40 to 55 (FIG. 7).

The difference between allele specific CNVs among different ethnicities show that >90% of the European and African-American samples have differences within 10. However, the Hispanic population shows a different pattern where ˜90% of the samples have differences between 10 and 30.

### Methods

Estimating KIV-2 CNV

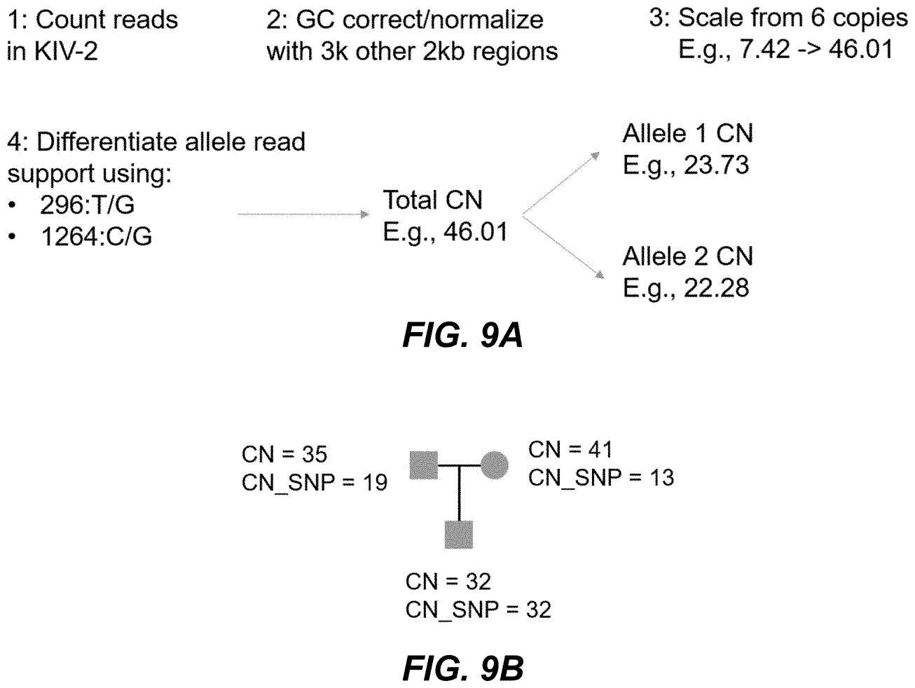

Referring to FIG. 9A, a method of determining the KIV-2 domain total CN and phasing (or determining) the KIV-2 domain CNs of the two alleles of a subject can include: 1. Counting reads aligned or mapped to the KIV-2 region of LPA gene in a reference sequence, such as a reference genome sequence. The KIV-2 region can include, for example, 6 copies of the KIV-2 domain in the reference sequence. The method can include: 2. Normalizing and GC correcting with 3 k other 2 kb regions. The normalized and GC corrected reads can be (or indicate) the number of the KIV-2 region (e.g., 7.42 illustrated in FIG. 9A). The method can include: 3. Scaling from the number of copies of the KIV-2 region by the number of copies of the KIV-2 domain in the KIV-2 region in the reference sequence, such as 6 copies, to determine the total copy number of the KIV-2 domain in the reference sequence (e.g., 46.01). The scaling factor can be based on the number of copies of KIV-2 domain in the KIV-2 region. For example, the scaling factor can be the number of copies of the KIV-2 domain in the KIV-2 region of LPA gene adjusted (e.g., multiplied by) a correction factor. For example, the scaling factor can be 6.2 when the number of copies of the KIV-2 domain in the KIV-2 region is 6. The correction factor can correct for sequencing bias. The correction factor can be empirically determined. To phase the KIV-2 CNs of the two alleles of a subject, the method can include: 4. Differentiating reads support the alleles using T/G at position 296 and C/G at position 1264 of a KIV-2 domain. These two single-nucleotide variants (SNVs) are always present on all copies of the KIV2 repeat of an allele if any copies. If present on one and only one allele, these two SNVs can be used to determine the proportion of total copy number belonging to each inherited allele, thus inferring the size of each allele. For example, a person can have one allele with T and C at position 296 and position 1264 of all copies of the KIV-2 domain and another allele with G and G at position 296 and position 1264 of all copies of the KIV-2 domain. Reads (or the percentage of the reads) with T and C at these two positions and reads with G and G at these two positions can be used to determine the copy number of the KIV-2 domain of each allele (e.g., 23.73 and 22.28). For example, 51.57% of the reads can have T and C at these two positions, and 48.43% of the reads can have G and G at these two positions. If the total copy number of the KIV-2 domain in the KIV-2 region of the LPA gene determined for a subject is 46.01, then based on the reads (or the percentage of the reads) with T and C at these positions and G and G at these positions, the copy number of the KIV-2 domain of the two alleles of the subject can be determined to be 23.73 and 22.28 copies (e.g., 46.01*51.57% equals 23.73, 46.01*48.43% equals 22.28).

Referring to FIG. 9B, one parent (father) of a person (male) has one allele with 16 copies of the KIV-2 domain and T and C at these positions and another allele with 19 copies of the KIV-2 domain and G and G at these positions. The other parent (mother) of the person has one allele with 28 copies of the KIV-2 domain and T and C at these positions and another allele with 13 copies of the KIV-2 domain and G and G at these positions. As illustrated in FIG. 9B, the person can inherit the allele with 19 copies of the KIV-2 domain and G and G at these positions from one parent (father) and the allele with 13 copies of the KIV-2 domain and G and G at these positions (mother) such that the two alleles of the person can have a total of 32 copies of the KIV-2 domain with G and G at these positions. The copy number of the KIV-2 domain of each allele of the person can be determined based on the copy number of the KIV-2 domain and the particular nucleobases at these positions of each allele of each parent. It is also possible that the person can inherit the allele with 19 copies of the KIV-2 domain and G and G at these positions from one parent (father) and the allele with 28 copies of the KIV-2 domain and T and C at these positions (mother). The copy number of the KIV-2 domain of each allele of the person can then be determined based on the reads with T and C at these positions and G and G at these positions. The copy number of the KIV-2 domain of each allele determined can be confirmed with (or checked using) the copy number of the KIV-2 domain and the particular nucleobases at these positions of each allele of each parent. It is also possible that the person can inherit the allele with 16 copies of the KIV-2 domain and T and C at these positions from one parent (father) and the allele with 13 copies of the KIV-2 domain and G and G at these positions (mother). The copy number of the KIV-2 domain of each allele determined can be confirmed with (or checked using) the copy number of the KIV-2 domain and the particular nucleobases at these positions of each allele of each parent. It is possible that the person can inherit the allele with 16 copies of the KIV-2 domain and T and C at these positions from one parent (father) and the allele with 28 copies of the KIV-2 domain and T and C at these positions (mother) such that the two alleles of the person can have a total of 44 copies of the KIV-2 domain with T and C at these positions. The copy number of the KIV-2 domain of each allele of the person can be determined based on the copy number of the KIV-2 domain and the particular nucleobases at these positions of each allele of each parent.

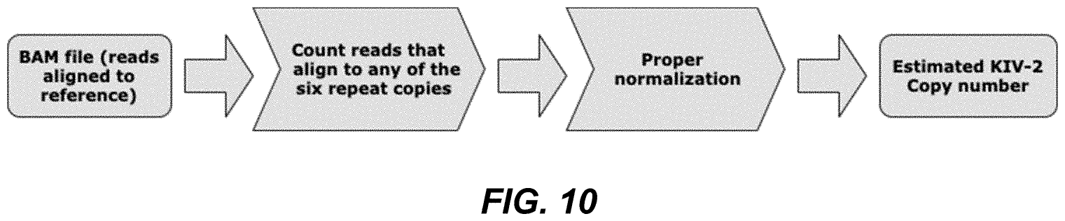

Illumina whole-genome sequencing (WGS) BAM files were used to measure the copy number for KIV-2 using the Kiv2CN methods disclosed herein (FIG. 10). The number of reads aligned to the KIV-2 region were counted, incorporating the six copies included within the standard GRCh37 or GRCh38 human reference genomes. Reads were then counted from 3,000 additional regions for normalization, each 3,000 bases in length. For quality control, the median absolute deviation (MAD) score was calculated and used to flag samples with high variation in coverage, with a maximum MAD threshold of 0.11. In these cases, KIV-2 copy number was still reported but, in some embodiments, with lower accuracy.

Read counts for all regions were normalized by region length, then by GC content using LOWESS regression. This smoothing method utilizes read counts from the set of normalization regions with their GC contents to predict the best adjustment for the read coverage of the KIV-2 region. The resulting normalized KIV-2 coverage metric, representing the number of copies of the entire KIV-2 region as represented by the reference genome, was then scaled by six to represent instead the number of copies of the KIV-2 repeat unit. This scaled value is the total copy number of KIV-2 in the sample, regardless of allele phase.

To refine the total copy number call and identify the allelic copy numbers, kiv2CN then counted reads aligning to two common intronic SNPs (T>G at chr6:160630428/160635977/160641520/160624884/160619338/160613786 and C>G at chr6:160620306/160625852/160631396/160636945/160642488/160614754, hg38), positions 296 and 1,264 within the repeat unit. These SNPs occur concomitantly and, most importantly, they occur in every copy of the repeat if present in any. In some embodiments where they occur on one allele of KIV-2 and not the other (the paternal copy only or the maternal copy only), they can be used to differentiate the proportion of KIV-2 total copy number derived from each allele. Therefore, the ratio of reads supporting the differentiating SNPs to those supporting the reference bases at those sites were calculated. In some embodiments, if at least ten reads support both reference and alternate alleles at both sites, this ratio was multiplied against the KIV-2 total copy number already determined. The result is the allelic copy number for one allele, and the remainder is the allelic copy number for the other. The total copy number and allelic copy numbers were then reported. This strategy ensured that the total copy number was reported for all samples, with allelic copy numbers also being found for ˜40% due to the prevalence of the differentiating SNPs used for allelic copy number estimation.

KIV-2 Assembly with PacBio HiFi Reads

The length of the KIV-2 repeat unit (˜5.5 kb) and often high allelic copy number (with a median of 15 copies per allele in the 1 KG cohort) can create extremely long single KIV-2 repeat alleles. This greatly complicates full assembly, even when PacBio HiFi reads are available. Even high-quality whole-genome assemblies from the Telomere-to-Telomere Consortium (T2T) often result in large gaps within the region (FIG. 11). To overcome this difficulty, a targeted process for reconstruction of the KIV-2 allele with HiFi data was designed.

First a regional reference genome was designed including two 100 kb flanking regions on either side of the KIV-2 repeat coordinates and a single consensus sequence consisting of the six known reference copies of KIV-2 from GRCh38, collapsed together. HiFi reads previously aligned to the KIV-2 region or its flanks were extracted from a BAM file, the reads were converted to FASTQ format with samtools and reads were realigned to the KIV-2 consensus reference. In most cases these realigned reads included multiple sequential alignments to the consensus reference genome, indicating multiple copies of the KIV-2 repeat. However, a single HiFi read was generally only long enough to span 2-3 transitions between copies of the KIV-2 repeat, providing evidence for at most 4 distinct copies. Therefore, read-supported nodes were manually assembled (multiple transitions between KIV-2 copies differentiated by distinct single nucleotide variants (SNVs)) into full-allele assemblies, connecting reads with partial overlap of the upstream flank, to reads entirely internal to KIV-2, until reaching reads with partial overlap of the downstream flank.

Manual assembly allowed copies of the repeat identified as errors to be discarded by low read support and mutual exclusivity with other copies. In some embodiments, such as the multiple repetition of identical KIV-2 copies, this procedure may be very difficult or even impossible, but it allowed 16 alleles in publicly available samples from the HPRC to be reconstructed.

### Determining CN of KIV-2 Domain of LPA Gene

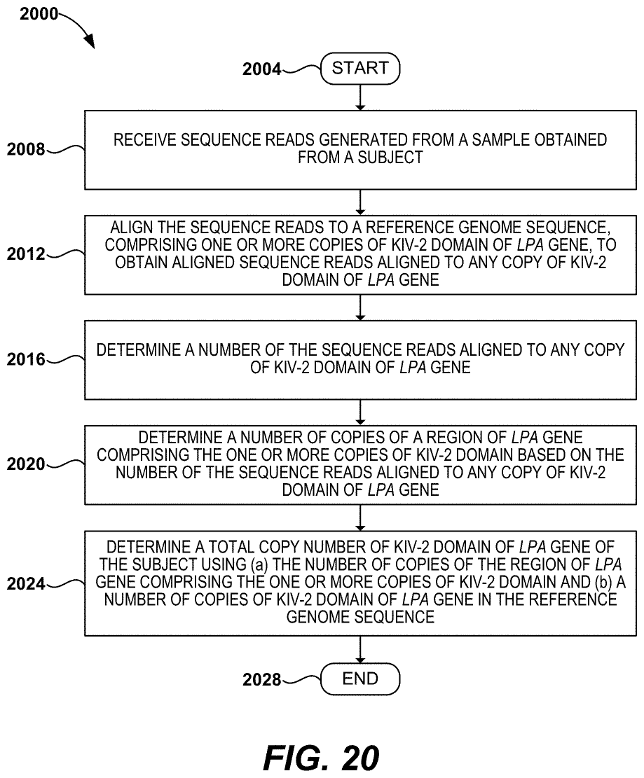

FIG. 20 is a flow diagram showing an exemplary method 2000 of determining a copy number of kringle IV type 2 (KIV-2) domain of LPA gene. The method 2000 is a targeted approach of determining CNs of the KIV-2 domain of LPA gene. LPA gene encodes apolipoprotein (apo(a)), a component of the complex particle lipoprotein (a) (Lp(a)). The method 2000 can be used to determine the total CN of the KIV-2 domain of LPA gene of a subject and/or to phase (or determine) the CN of the KIV-2 domain of each allele of LPA gene of the subject as described herein. The method 2000 can have the performance described herein. For example, the method 2000 can have high accuracy (See FIGS. 3A-3B and 4A-4B and accompanying descriptions). The method 2000 can resolve the challenge of KIV-2 copy number identification. The method 2000 can accurately determine (or estimate) KIV-2 copy number. The method 2000 can provide a more refined estimate of the allelic copy number of KIV-2. The method 2000 can be used to generate the data and results described herein, such as those illustrated in FIGS. 3A-3B, 4A-4B, 5-8, and 11-19.

Lp(a) is the strongest independent genetic cardiovascular disease (CVD) risk factor. High Lp(a) levels (e.g., greater 30 mg/dL) increase CVD risk by 2×-4×. About 1/5 individuals have elevated Lp(a) levels. At-risk patients are typically asymptomatic until first coronary events. Plasma Lp(a) concentration is largely determined by LPA's kringle IV-2 (KIV-2) domain. The KIV-2 domain of LPA gene is a Variable Number of Tandem Repeats (VNTR). A reference genome sequence can include 6 copies of the KIV-2 domain of LPA gene. KIV-2 repeat accounts for majority of variability (˜69%). KIV-2 copy number is inversely correlated with Lp(a) concentration. A decrease in KIV-2 repeat count correlates with an increase in plasma Lp(a) level. Risk of myocardial infarction increases with Lp(a) concentration (low KIV-2 copy number). Longer isoforms of Lp(a) often are not secreted by endoplasmic reticulum. Common genome-wide copy-number variant calling strategies fail to accurately call VNTR variations.

The human LPA gene encodes apolipoprotein (apo(a)), a component of the complex particle lipoprotein (a) (Lp(a)). High concentrations of Lp(a) in blood plasma have been associated with increased risk of coronary heart disease, thus accurate assessment of genetic factors that influence Lp(a) levels are essential. A specific domain of the LPA gene, known as kringle IV-2 (KIV-2) is highly variable in copy number and can have a major impact on the total length of the resulting apo(a) transcript. The impact on transcript length in turn impacts the production of finished Lp(a) in blood plasma, likely due to overly long transcripts being retained in the endoplasmic reticulum. The length of each genomic copy of LPA, specifically the number of copies of the KIV-2 repeat (including one paternal and one maternal copy) is therefore a predictor of coronary heart disease risk.

Detecting the allelic copy number of KIV-2 is a challenging problem due to its high variability, the similarity of individual copies and the length of the repeat unit. The KIV-2 repeat consists of 5.5 kilobases (kb) of genomic material, and often ranges from 5 to >30 copies, including copies that sequentially be identical or nearly identical. Thus sequencing approaches with short reads fail to span a single repeat unit, and even long-read sequencing struggles to span the entire locus to recover full allele length. Detecting (or determining) KIV-2 copy number from short-read sequencing (and even long-read or genome-mapping) can be challenging. Existing methods for detecting KIV-2 copy number often struggles to accurately resolve the full repeat array.

The method 2000 can resolve the challenge of KIV-2 copy number identification. It applies a depth-based counting strategy using, for example, Illumina short-read sequencing across whole genomes to accurately estimate KIV-2 copy number. Reads that align to the KIV-2 region of the reference genome, which includes six copies of the repeat unit, are identified and counted. Reads are also counted from an additional selection of 3,000 distinct and diverse 2 kb regions of the genome. The read counts from each region are normalized to the length of the region, then pooled and normalized across all regions to correct for differences in sequencing depth and sequencing bias resulting from proportion of the nucleotides G and C. The normalized depth metric for the KIV-2 region can be scaled to the number of reference copies of the KIV-2 domain in the KIV-2 region. The scaled and normalized total represents a highly accurate estimate of the total number of copies of the KIV-2 repeat. Unlike long-read or genome mapping approaches which attempt to span the full repeat region, the success of this approach is independent of allele length or sequence identity between sequential copies, meaning that total copy number can be reported, in some embodiments, in most or all situations.

The method 2000 can provide a more refined estimate of the allelic copy number of KIV-2 in many cases, based on a pair of common intronic single-nucleotide variants (SNVs). These SNPs are present in all or nearly all copies of the KIV-2 repeat array if any, and therefore can be used to differentiate between allelic origins of reads if exactly one inherited copy of the KIV-2 allele contains them. This variant caller can therefore report allelic copy number in many cases as well as total copy number.

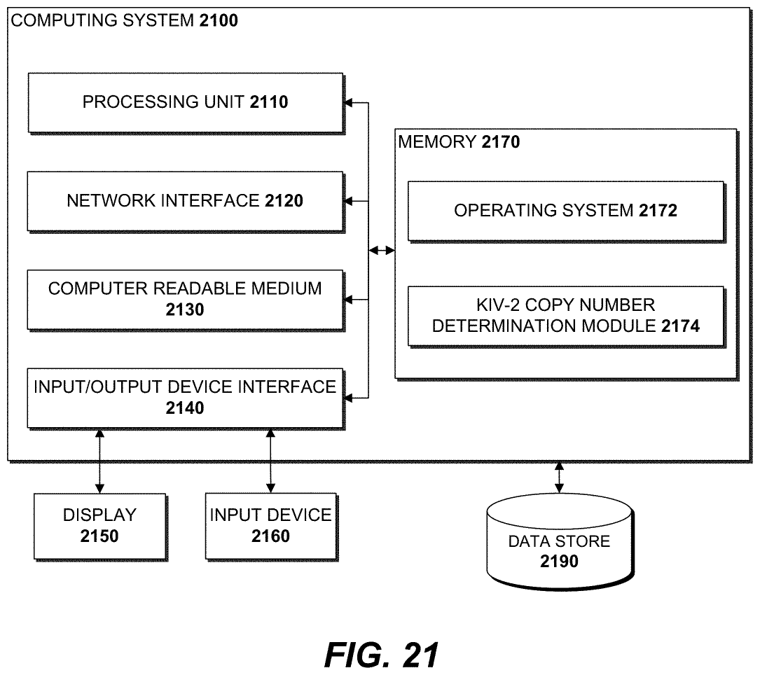

The method 2000 may be embodied in a set of executable program instructions stored on a computer-readable medium, such as one or more disk drives, of a computing system. For example, the computing system 2100 shown in FIG. 21 and described in greater detail below can execute a set of executable program instructions to implement the method 2000. When the method 2000 is initiated, the executable program instructions can be loaded into memory, such as RAM, and executed by one or more processors of the computing system 2100. Although the method 2000 is described with respect to the computing system 2100 shown in FIG. 21, the description is illustrative only and is not intended to be limiting. In some embodiments, the method 2000 or portions thereof may be performed serially or in parallel by multiple computing systems.

After the method 2000 begins at block 2004, the method 2000 proceeds to block 2008, where a computing system (e.g., the computing system 2100 described with reference to FIG. 21) receives a plurality of sequence reads. The plurality of sequence reads can be generated from a sample obtained from a subject (which can be a mammal, such as a human). The sample can be obtained directly from the subject. The sample can be generated from another sample obtained from a subject. The other sample can be obtained directly from the subject. The computing system can store the plurality of sequence reads in its memory. The computing system can load the plurality of sequence reads into its memory. Sequence reads can be generated by techniques such as sequencing by synthesis, sequencing by binding, or sequencing by ligation. Sequence reads can be generated using instruments such as MINISEQ, MISEQ, NEXTSEQ, HISEQ, and NOVASEQ sequencing instruments from Illumina, Inc. (San Diego, CA).

Sequence reads can be, for example, 50, 60, 70, 80, 90, 100, 110, 120, 130, 140, 150, 160, 170, 180, 190, 200, 300, 400, 500, 600, 700, 800, 900, 1000, 1250, 1500, 1750, 2000, or more base pairs (bps) in length each. For example, sequence reads are about 100 base pairs to about 1000 base pairs in length each. The sequence reads can comprise paired-end sequence reads. The sequence reads can comprise single-end sequence reads. The sequence reads can be generated by whole genome sequencing (WGS). The WGS can be clinical WGS (cWGS). The sequence reads can comprise single-end sequence reads. The sequence reads can be generated by targeted sequencing, such as sequencing of 5, 10, 20, 30, 40, 50, 100, 200, or more genes. The sample can comprise cells, cell-free DNA, cell-free fetal DNA, amniotic fluid, a blood sample, a biopsy sample, or a combination thereof.

The method 2000 proceeds from block 2008 to block 2012, where the computing system aligns the plurality of sequence reads to a reference sequence (or a reference genome sequence), comprising one or more copies of the KIV-2 domain of the LPA gene, to obtain a plurality of aligned sequence reads comprising sequence reads aligned to any copy of the KIV-2 domain of the LPA gene in the reference genome sequence. The reference sequence can be a reference human genome sequence, such as hg38 or hg19, or a portion thereof.

A sequence read of the sequence reads aligned to any copy of the KIV-2 domain of the LPA gene in the reference sequence can have a low alignment quality score. The computing system can align sequence reads to the reference sequence using an aligner or an alignment method such as Burrows-Wheeler Aligner (BWA), ISAAC, BarraCUDA, BFAST, BLASTN, BLAT, Bowtie, CASHX, Cloudburst, CUDA-EC, CUSHAW, CUSHAW2, CUSHAW2-GPU, drFAST, ELAND, ERNE, GNUMAP, GEM, GensearchNGS, GMAP and GSNAP, Geneious Assembler, LAST, MAQ, mrFAST and mrsFAST, MOM, MOSAIK, MPscan, Novoaligh & NovoalignCS, NextGENe, Omixon, PALMapper, Partek, PASS, PerM, PRIMER, QPalma, RazerS, REAL, cREAL, RMAP, rNA, RT Investigator, Segemehl, SeqMap, Shrec, SHRiMP, SLIDER, SOAP, SOAP2, SOAP3 and SOAP3-dp, SOCS, SSAHA and SSAHA2, Stampy, SToRM, Subread and Subjunc, Taipan, UGENE, VelociMapper, XpressAlign, and ZOOM.

The method 2000 proceeds from block 2012 to block 2016, where the computing system determines a number of the sequence reads aligned to any copy of the KIV-2 domain of the LPA gene in the reference sequence. For example, read counts for all regions can be normalized by region length, then by GC content. The number of reads aligned to the KIV-2 region, which includes the one or more copies of the KIV-2 domain, such as six KIV-2 domains, can be determined. The number of the sequence reads aligned to any copy of the KIV-2 domain of the LPA gene in the reference sequence can comprise a raw number or a normalized and/or GC-corrected number of the sequence reads aligned to any copy of the KIV-2 domain of the LPA gene in the reference sequence.

The computing system can determine the normalized number of the sequence reads aligned to any copy of the KIV-2 domain of the LPA gene in the reference sequence. For example, the computing system can determine the normalized number of the sequence reads aligned to any copy of the KIV-2 domain of the LPA gene in the reference sequence using (1a) a depth of the sequence reads aligned to any copy of the KIV-2 domain of the LPA gene in the reference sequence. The computing system can determine the normalized number of the sequence reads aligned to any copy of the KIV-2 domain of the LPA gene in the reference sequence using (1b) a length of the region of the LPA gene in the reference sequence comprising the one or more copies of the KIV-2 domain. The region of the LPA gene in the reference sequence comprising the one or more copies of the KIV-2 domain can be referred to as the KIV-2 region. The computing system can determine the normalized number of the sequence reads aligned to any copy of the KIV-2 domain of the LPA gene in the reference sequence using (2a) a depth of sequence reads of the plurality of sequence reads aligned to each of a plurality of regions of the reference sequence other than a genetic locus comprising LPA gene. The computing system can determine the normalized number of the sequence reads aligned to any copy of the KIV-2 domain of the LPA gene in the reference sequence using (2b) a length of each of the plurality of regions of the reference genome other than the genetic locus comprising LPA gene.

The computing system can determine the GC corrected number of the sequence reads aligned to any copy of the KIV-2 domain of the LPA gene in the reference sequence from the number or the normalized number of the sequence reads aligned any copy of the KIV-2 domain of the LPA gene in the reference sequence. For example, the computing system can determine the GC corrected number of the sequence reads aligned to any copy of the KIV-2 domain of the LPA gene in the reference sequence from the number or the normalized number of the sequence reads aligned any copy of the KIV-2 domain of the LPA gene in the reference sequence using a GC content of the region of the LPA gene in the reference sequence comprising the one or more copies of the KIV-2 domain.

The method 2000 proceeds from block 2016 to block 2020, where the computing system determines a number of copies of the region of the LPA gene comprising the one or more copies of the KIV-2 domain based on the number of the sequence reads aligned to any copy of the KIV-2 domain of the LPA gene in the reference sequence. For example, the number of copies (e.g., 7.42) of the entire KIV-2 region as represented by the reference sequence can be determined. The computing system can determine the number of copies of the region of the LPA gene comprising the one or more copies of the KIV-2 domain using a normalized and/or GC-corrected number of the sequence reads aligned to any copy of the KIV-2 domain of the LPA gene in the reference sequence.

The method 2000 proceeds from block 2020 to block 2024, where the computing system determines a total copy number (e.g., 44.52) of the KIV-2 domain of the LPA gene of the subject using (a) the number of copies (e.g., 7.42) of the region of the LPA gene comprising the one or more copies of the KIV-2 domain and (b) a number of copies of the KIV-2 domain of the LPA gene in the reference sequence (e.g., 6 copies). The number of copies of the KIV-2 domain of the LPA gene in the reference sequence can be, for example, 2, 3, 4, 5, 6, 7, 8, 9, 10, 20, 30, 40, 50, or more copies. For example, the number of copies (e.g., 7.42) of the entire KIV-2 region can be scaled by a scaling factor to determine the total copy number (e.g., 44.52) of the KIV-2 domain of the LPA gene. The scaling factor can be based on the number of the copies of the KIV-2 domain of the LPA gene in the reference sequence. For example, the number of the copies of the KIV-2 domain of the LPA gene in the reference sequence can be 6, and the scaling factor can be about 6. In some embodiments, the scaling factor is the number of the copies of the KIV-2 domain of the LPA gene in the reference genome sequence adjusted by a correction factor. The scaling factor can be the number of the copies of the KIV-2 domain of the LPA gene in the reference genome sequence multiplied by a correction factor. For example, the number of the copies of the KIV-2 domain of the LPA gene in the reference sequence can be 6, and the scaling factor can be 6.2 with the correction factor being 1 and 1/33. The correction factor can be 1.01 to 1.2 (or about 1.01 to about 1.2), such as (about) 1.01, 1.02, 1.03, 1.033, 1.04, 1.05, 1.06, 1.07, 1.08, 1.09, 1.1, 1.11, 1.12, 1.13, 1.14, 1.15, 1.16, 1.17, 1.18, 1.19, or 1.2. The correction factor can correct for sequencing bias. The correction factor can be predetermined. The correction factor can be empirically determined. In some embodiments, the scaling factor is the number of the copies of the KIV-2 domain of the LPA gene in the reference genome sequence. To determine the total copy number of the KIV-2 domain of the LPA gene of the subject, the computing system can scale (e.g., multiply) the number of copies of the region of the LPA gene comprising the one or more copies of the KIV-2 domain by the number of the copies of the KIV-2 domain of the LPA gene in the reference sequence to determine the total copy number of the KIV-2 domain of the LPA gene of the subject.

In some embodiments, the computing system can determine (a) a number of copies of the KIV-2 domain of the LPA gene of a first allele of the subject and (b) a number of copies of the KIV-2 domain of the LPA gene of a second allele of the subject. For example, to refine the total copy number call and identify the allelic copy numbers, the computing system can count reads aligning to two common intronic SNPs. The computing system can determine (a) a number of copies of the KIV-2 domain of the LPA gene of a first allele of the subject and (b) a number of copies of the KIV-2 domain of the LPA gene of a second allele of the subject based on one or more single nucleotide variants (SNVs) of the KIV-2 domain of the LPA gene. The one or more SNVs comprise T>G at position 296 and C>G at position 1264 of a copy of the KIV-2 domain of the LPA gene in the reference sequence. The copy of the KIV-2 domain can comprise a sequence of SEQ ID NO: 1 (chr6:160613491-160619042 of hg38). The one or more SNVs comprise T>G at chr6:160630428, 160635977, 160641520, 160624884, 160619338, and/or 160613786 of hg38 and/or C>G at chr6:160620306, 160625852, 160631396, 160636945, 160642488, and/or 160614754 of hg38 or at corresponding positions of another reference genome sequence (e.g., hg19).

In some embodiments, the computing system can create a file or a report and/or generate a user interface (UI) comprising a UI element representing or comprising (i) the total copy number of the KIV-2 domain of the LPA gene of the subject and/or (iia) a number of copies of the KIV-2 domain of the LPA gene of a first allele of the subject and (iib) a number of copies of the KIV-2 domain of the LPA gene of a second allele of the subject. A UI element can be a window (e.g., a container window, browser window, text terminal, child window, or message window), a menu (e.g., a menu bar, context menu, or menu extra), an icon, or a tab. A UI element can be for input control (e.g., a checkbox, radio button, dropdown list, list box, button, toggle, text field, or date field). A UI element can be navigational (e.g., a breadcrumb, slider, search field, pagination, slider, tag, icon). A UI element can informational (e.g., a tooltip, icon, progress bar, notification, message box, or modal window). A UI element can be a container (e.g., an accordion).

In some embodiments, the computing system can determine a likely concentration of Lipoprotein(a) in the subject using the total copy number of the KIV-2 domain of the LPA gene of the subject. The computing system can determine a likelihood of myocardial infarction and/or coronary arterial disease in the subject using the total copy number of the KIV-2 domain of the LPA gene of the subject and/or the likely concentration of Lipoprotein(a) in the subject.

The method 2000 ends at block 2028.

### Execution Environment

FIG. 21 depicts a general architecture of an example computing device 2100 configured to execute the processes and implement the features described herein. The general architecture of the computing device 2100 depicted in FIG. 21 includes an arrangement of computer hardware and software components. The computing device 2100 may include many more (or fewer) elements than those shown in FIG. 21. It is not necessary, however, that all of these generally conventional elements be shown in order to provide an enabling disclosure. As illustrated, the computing device 2100 includes a processing unit 2110, a network interface 2120, a computer readable medium drive 2130, an input/output device interface 2140, a display 2150, and an input device 2160, all of which may communicate with one another by way of a communication bus. The network interface 2120 may provide connectivity to one or more networks or computing systems. The processing unit 2110 may thus receive information and instructions from other computing systems or services via a network. The processing unit 2110 may also communicate to and from memory 2170 and further provide output information for an optional display 2150 via the input/output device interface 2140. The input/output device interface 2140 may also accept input from the optional input device 2160, such as a keyboard, mouse, digital pen, microphone, touch screen, gesture recognition system, voice recognition system, gamepad, accelerometer, gyroscope, or other input device.

The memory 2170 may contain computer program instructions (grouped as modules or components in some embodiments) that the processing unit 2110 executes in order to implement one or more embodiments. The memory 2170 generally includes RAM, ROM and/or other persistent, auxiliary or non-transitory computer-readable media. The memory 2170 may store an operating system 2172 that provides computer program instructions for use by the processing unit 2110 in the general administration and operation of the computing device 2100. The memory 2170 may further include computer program instructions and other information for implementing aspects of the present disclosure.

For example, in one embodiment, the memory 2170 includes a KIV-2 copy number determination module 2174 for determining the copy number (e.g., total copy number or the copy number of each allele) of the KIV-2 domain of the LPA gene a subject has, such as the method 2000 described with reference to FIG. 20. In addition, memory 2170 may include or communicate with the data store 2190 and/or one or more other data stores that store the input and/or output of the method 2000, such as the plurality of sequence reads generated from a sample obtained from a subject, the total copy number of the KIV-2 domain of the LPA gene of the subject, and/or the copy number of the KIV-2 domain in each allele of the LPA gene of the subject.

### Additional Considerations

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

One skilled in the art will appreciate that, for this and other processes and methods disclosed herein, the functions performed in the processes and methods can be implemented in differing order. Furthermore, the outlined steps and operations are only provided as examples, and some of the steps and operations can be optional, combined into fewer steps and operations, or expanded into additional steps and operations without detracting from the essence of the disclosed embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Accordingly, phrases such as “a device configured to” are intended to include one or more recited devices. Such one or more recited devices can also be collectively configured to carry out the stated recitations. For example, “a processor configured to carry out recitations A, B and C can include a first processor configured to carry out recitation A and working in conjunction with a second processor configured to carry out recitations B and C. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

It is to be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein.

All of the processes described herein may be embodied in, and fully automated via, software code modules executed by a computing system that includes one or more computers or processors. The code modules may be stored in any type of non-transitory computer-readable medium or other computer storage device. Some or all the methods may be embodied in specialized computer hardware.

Many other variations than those described herein will be apparent from this disclosure. For example, depending on the embodiment, certain acts, events, or functions of any of the algorithms described herein can be performed in a different sequence, can be added, merged, or left out altogether (for example, not all described acts or events are necessary for the practice of the algorithms). Moreover, in certain embodiments, acts or events can be performed concurrently, for example through multi-threaded processing, interrupt processing, or multiple processors or processor cores or on other parallel architectures, rather than sequentially. In addition, different tasks or processes can be performed by different machines and/or computing systems that can function together.

The various illustrative logical blocks and modules described in connection with the embodiments disclosed herein can be implemented or performed by a machine, such as a processing unit or processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, for example a combination of a DSP and a microprocessor, a plurality of microprocessors, one or more microprocessors in conjunction with a DSP core, or any other such configuration. Although described herein primarily with respect to digital technology, a processor may also include primarily analog components. For example, some or all of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

Any process descriptions, elements or blocks in the flow diagrams described herein and/or depicted in the attached figures should be understood as potentially representing modules, segments, or portions of code which include one or more executable instructions for implementing specific logical functions or elements in the process. Alternate implementations are included within the scope of the embodiments described herein in which elements or functions may be deleted, executed out of order from that shown, or discussed, including substantially concurrently or in reverse order, depending on the functionality involved as would be understood by those skilled in the art.

It should be emphasized that many variations and modifications may be made to the above-described embodiments, the elements of which are to be understood as being among other acceptable examples. All such modifications and variations are intended to be included herein within the scope of this disclosure and protected by the following claims.

1. A method for determining a copy number of kringle IV type 2 (KIV-2) domain of LPA gene comprising:
under control of a hardware processor:
receiving a plurality of sequence reads generated from a sample obtained from a subject;
aligning the plurality of sequence reads to a reference genome sequence, comprising one or more copies of the KIV-2 domain of the LPA gene, to obtain a plurality of aligned sequence reads comprising sequence reads aligned to any copy of the KIV-2 domain of the LPA gene in the reference genome sequence;
determining a number of the sequence reads aligned to any copy of the KIV-2 domain of the LPA gene in the reference genome sequence;
determining a number of copies of a region of the LPA gene comprising the one or more copies of the KIV-2 domain based on the number of the sequence reads aligned to any copy of the KIV-2 domain of the LPA gene in the reference genome sequence; and
determining a total copy number of the KIV-2 domain of the LPA gene of the subject using (a) the number of copies of the region of the LPA gene comprising the one or more copies of the KIV-2 domain and (b) a number of copies of the KIV-2 domain of the LPA gene in the reference genome sequence.

2. (canceled)

3. (canceled)

4. (canceled)

5. (canceled)

6. The method of claim 1, wherein the number of the sequence reads aligned to any copy of the KIV-2 domain of the LPA gene in the reference sequence comprises a raw number or a normalized and/or GC-corrected number of the sequence reads aligned to any copy of the KIV-2 domain of the LPA gene in the reference sequence.

7. The method of claim 1, wherein determining the number of copies of the region of the LPA gene comprising the one or more copies of the KIV-2 domain comprises: determining the number of copies of the region of the LPA gene comprising the one or more copies of the KIV-2 domain using a normalized and/or GC-corrected number of the sequence reads aligned to any copy of the KIV-2 domain of the LPA gene in the reference genome sequence.

8.-18. (canceled)

19. A system for determining a copy number of kringle IV type 2 (KIV-2) domain of LPA gene comprising:
non-transitory memory configured to store executable instructions and a plurality of sequence reads generated from a sample obtained from a subject; and
a hardware processor in communication with the non-transitory memory, the hardware processor programmed by the executable instructions to perform:
aligning the plurality of sequence reads to a reference sequence, comprising one or more copies of the KIV-2 domain of the LPA gene, to obtain a plurality of aligned sequence reads comprising sequence reads aligned to any copy of the KIV-2 domain of the LPA gene in the reference sequence;
determining a number of the sequence reads aligned to any copy of the KIV-2 domain of the LPA gene in the reference sequence;
determining a normalized, GC-corrected number of the sequence reads aligned to any copy of the KIV-2 domain of the LPA gene in the reference sequence;
determining a number of copies of a region of the LPA gene comprising the one or more copies of the KIV-2 domain using the normalized, GC-corrected number of the sequence reads aligned to any copy of the KIV-2 domain of the LPA gene in the reference sequence; and
determining a total copy number of the KIV-2 domain of the LPA gene of the subject using (a) the number of copies of the region of the LPA gene comprising the one or more copies of the KIV-2 domain and (b) a number of copies of the KIV-2 domain of the LPA gene in the reference sequence.

20. The system of claim 19, wherein the reference sequence comprises a reference genome sequence.

21. The system of claim 19, wherein the hardware processor is further programmed by the executable instructions to perform: determining (a) a number of copies of the KIV-2 domain of the LPA gene of a first allele of the subject and (b) a number of copies of the KIV-2 domain of the LPA gene of a second allele of the subject, based on one or more single nucleotide variants (SNVs) of the KIV-2 domain of the LPA gene.

22. The system of claim 19, wherein the one or more SNVs comprise T>G at position 296 and C>G at position 1264 of a copy of the KIV-2 domain of the LPA gene in the reference genome sequence, optionally wherein the copy of the KIV-2 domain comprises a sequence of SEQ ID NO: 1.

23. The system of claim 19, wherein the one or more SNVs comprise G>T at chr6:160630428, 160635977, 160641520, 160624884, 160619338, and/or 160613786 of hg38 and/or G>C at chr6:160620306, 160625852, 160631396, 160636945, 160642488, and/or 160614754 of hg38 or at corresponding positions of another reference genome sequence.

24. The system of claim 19, wherein a sequence read of the sequence reads aligned to any copy of the KIV-2 domain of the LPA gene in the reference genome sequence with a low alignment quality score.

25. The system of claim 19, wherein determining the normalized, GC-corrected number of the sequence reads aligned to any copy of the KIV-2 domain of the LPA gene in the reference genome sequence comprises: determining the normalized number of the sequence reads aligned to any copy of the KIV-2 domain of the LPA gene in the reference genome sequence using (1a) a depth of the sequence reads aligned to any copy of the KIV-2 domain of the LPA gene in the reference genome sequence, (1b) a length of the region of the LPA gene in the reference genome sequence comprising the one or more copies of the KIV-2 domain, (2a) a depth of sequence reads of the plurality of sequence reads aligned to each of a plurality of regions of the reference genome sequence other than a genetic locus comprising LPA gene, and (2b) a length of each of the plurality of regions of the reference genome other than the genetic locus comprising LPA gene.

26. The system of claim 25, wherein determining the normalized, GC-corrected number of the sequence reads aligned to any copy of the KIV-2 domain of the LPA gene in the reference genome sequence comprises: determining the normalized, GC-corrected number of the sequence reads aligned to any copy of the KIV-2 domain of the LPA gene in the reference genome sequence from the normalized number of the sequence reads aligned any copy of the KIV-2 domain of the LPA gene in the reference genome sequence using a GC content of the region of the LPA gene in the reference genome sequence comprising the one or more copies of the KIV-2 domain.

27. The system of claim 19, wherein determining the total copy number of the KIV-2 domain of the LPA gene of the subject comprises: scaling the number of copies of the region of the LPA gene comprising the one or more copies of the KIV-2 domain by a scaling factor to determine the total copy number of the KIV-2 domain of the LPA gene of the subject, wherein the scaling factor is based on the number of the copies of the KIV-2 domain of the LPA gene in the reference genome sequence, optionally wherein the scaling factor is the number of the copies of the KIV-2 domain of the LPA gene in the reference genome sequence adjusted by a correction factor, optionally wherein the correction factor is about 0.01 to about 0.1, and optionally wherein the scaling factor is the number of the copies of the KIV-2 domain of the LPA gene in the reference genome sequence.

28. The system of claim 19, wherein the number of copies of the KIV-2 domain of the LPA gene in the reference genome sequence is six.

29. The system of claim 19, wherein the hardware processor is further programmed by the executable instructions to perform: creating a file or a report and/or generating a user interface (UI) comprising a UI element representing or comprising (i) the total copy number of the KIV-2 domain of the LPA gene of the subject and/or (iia) a number of copies of the KIV-2 domain of the LPA gene of a first allele of the subject and (iib) a number of copies of the KIV-2 domain of the LPA gene of a second allele of the subject.

30. The system of claim 19, wherein the hardware processor is further programmed by the executable instructions to perform: determining a likely concentration of Lipoprotein(a) in the subject using the total copy number of the KIV-2 domain of the LPA gene of the subject.

31. The system of claim 19, wherein the hardware processor is further programmed by the executable instructions to perform: determining a likelihood of myocardial infarction and/or coronary arterial disease in the subject using the total copy number of the KIV-2 domain of the LPA gene of the subject and/or the likely concentration of Lipoprotein(a) in the subject.

32. The system of claim 19, wherein the plurality of sequence reads comprises sequence reads that are about 100 base pairs to about 1000 base pairs in length each.

33. The system of claim 19, wherein the plurality of sequence reads comprises paired-end sequence reads and/or single-end sequence reads.

34. The system of claim 19, wherein the plurality of sequence reads is generated by whole genome sequencing (WGS), optionally wherein the WGS is clinical WGS (cWGS).

35. The system of claim 19, wherein the sample comprises cells, cell-free DNA, cell-free fetal DNA, amniotic fluid, a blood sample, a biopsy sample, or a combination thereof.


SEQUENCE LISTING (1 sequence)

SEQ ID NO: 1 - DNA, 5552 bp, Homo sapiens
  source 1..5552: mol_type = genomic DNA
catgcaaatgcatctgtatctgtctagtctccgtatctctctcggtaggacttcatattctaatgtgggagttgcaacgaacatgtagttcttcagaaaacgcacgtcatccaggaggatagccgttccaggtcaaccctcacactctatgtactcaatacgtagtgaaaaggctctacctttcccacagaaaagcattgtgcaaatagtattactgtctgtcaggtttagtaggtttgtttctccaagacttctcaaagctgccctggaaaacttgctcccaggcaggatgcagtatctctagaaggggttcctaggcaggtccttctctcctgctctcagtctaccctctgctggccgcagctactccgggactgagggaaagggggatgagttgaaagaacagtgggacccatggcataaaggaagacggcagggtggactaagagatctgaagaggtcggacagctacggagcagcagagaaaaatgcattagggggcaaagcaggcgagtagtttgggctgcggggatcttgaagcattcgctcttccttatgcctcccaagaacgttgctccaacctcccagtatcctcacattcatgacctctggctgcctgagaaaatgggaaatgtattatgggccatggggttccaacactgtctctctagagactctagacttgtggcctagtctcaaccccagaccctccattccaggaagctggttcaatgagcaactggatgtataagagcctgagctcttgaaagacttctagttcttccttggcttccctcttttgaatgcatggactattcttattataccaaaaacctcagagttcaaacctttgtgattgctctaggatttgcaatatcccttcgaacccgcacagtacccttttattaatatgacacaccatcgtgcatgaggtaaagaaacttacaacggtacacttcgaattaatcccttcgtgatttatgctaccatttcaaattcttacctctacatatgctgaagttaaatatcttgctatcattttgtttcaagtagtcaactctcatttacaaagatatagaaatttgatcaagaaaatattttgcatttacatctatctacatttgccaattgctggattcttcattacgtgggctgcagaaagttcctttcttgttcaattcttcttctacctaattccttcgggtcacctctcttgttacataagcctgctgatttccaattcttacagtttccggtttctttctttggggcagtgcacaatatgcatttatccgacatggatgaccttcaaggcattttattgctggattcagcattcccggttgatctctttttgttcctgattatcattttaacaatattccctggtatctggcttccagtgtttctgaggtgaacacattggcaatttgtatcagtgtacttgtaggaagacacagctctttttcctccagtttctcgtgacaggtttcttcttattacatctttgccttgttaccttcgaactgtgatgtacaaaagtacatcatacaggttgggttttttttccgaaattccctatatgatcccttgagtttcacgattctgtgattttttttaatgttcttcgtaattgaaaagttatcagccatccttgattctggtatttgtttctattataaactcccttgctctttactgtgacttgcttcccacatgcaggaaatcttttcatattttcctacatgtctgtgaggctcgcttaattttatatttcactgtttcttcaggggtgcgcgtgtctgtgtgtgttagaacttgtgagtttctgcgtgtgtgtgagtatgggtgtgttttcagtttgtgtgtgtgtgtgtgtgtgtgtgtgtgtgtgtagctcattctgtaggttctctaaaattgtgtgttgggtaatgttttcctctgaaacgtctaatttgatcagccggccatacagtaatttttttcaattaagacatactttttttatactagctctttaatttaatttggctattctgtactgttaatttctcttgatatgatatctgctttcttgcatgttgtttttttaaatatttgaaatcagttatcatactgatatgtcaaattttcacgcaattctaatcatttctctcattcctgagcctgtttgtattacccgatttatttcctggtcatgggtcacacgaaacgcttttctacatgtagtcagattttaaatttaggactcactctgcggataccgcatagctgaggcaatcgactttgttgtttacttctaaagagagttgagatttcctctcacaggcacttcattgattggaaaccgatttgatgcttggacacttgctttccagctgtgcaaggggttgtctgcagctgactttattctagtaatattctaagtgtacctttaaattacggctgttctggggtctccactgaattggcaatgtgctccgtgaggactctctggtctagctgggaagaactccagtatcccagagtgctatgtgatctctctcttctttattgagcttcctgtgctcttgtacttgctgtttctcagtaaatgttctttgccacactttctcgtggaaccatttcctgtgcacaggcagcgtcatgtcaggccaacgactggaggagacttctatgcgtatttgtagagctcttttcctccacacaaaccagttcttatttctagcagacctagaaaaatttcagctaccacagtcatcccaaactccaatccctctcctccattcagcaagatggctacagtctgcttgggctccatctccttgctattcagtgcaggaagggtatccaggaagaaagccaaggcatctgcggagtggagcccatcattgcccctctctcaattctctaggtcctctaccatctgttgtcccacatgtagaaaccatttttccatgtctctggtagtcgcttgtggccaaaatattggccattgggtggtcagacccgggcttctctccatccacccatcccttttacttttcagcatccctctctgtgctgactctcatctgccttccttccttggatcttctcttgctgggaacctgtatcccttcaggccactggtacttaggaacgagtgtttttctgacaatcggaatatacgtagatatagaaacagatcaattcacctgtccataaataagcacctattctttcctcttgaacccagagagaaaaggcaaacgcaaccttcccttcaggaagtggaagccagaataacggaattccaactggaaagtctccattgacgtttagttggtgttgtggaagggtaatctaagtgtctggtcctttgtcattctgactttcttcataatgtgatctcctcacctccagatttcccttctgctcaatagaactaggaggaaggagagggagcttaacatttcccttctctcagacccttagctccaaggcaatcatcctgagacattttgctacgccatctgcatctgtcacaagttgagttcggagaactcagcttgagacacatctcttgtaacagaaacttcacttggccctttcttctcttatggtaaagaacaaagacatacgcatttgggtagtattctggggtccgactatgcgagtgtggtgtcatagatgaccaagcttggcaggttcttcctgtgacagtggtggagtatgtgcctcgataactctgtccattaccatggtagcactcctgcaccccaggcctttgctcagtcggtgctgaaatgaaaacacgggaaatcaagctgagtatctctgagcatagagaaacatgtgaagccatttgtgacacaaccagaaaggagtctatgagaattacgaccgttctcttttccttatccataggcagatggatgtgagaaaaccgaccaacaaacaaacaccaaagcaacagattcctcccattctagaactgtagtaagttcttggaaatatttcacctgacaccttagagaaacacaacagatcagattcctcattggaaagtgagctcacgaggtagcacctttctaggaaactctactgaatcttcatgcaaacggcaccgcatttggcaatgcgggctgcaggtggtaattggctgtcgggggcagagtttgggacagacatggaaattttgcatgtttccctagaccgttgatcatgaaagccaaggcctttggcttccgggccatggggcagaaaacggtcctatgtatctgactgaagacaaagacactttgcttctcagggatgtgtgcacgccaaaagcttcttcccttggaggagagagggaatcctctcatgccctgcatcctgaattgtagtaaagtaggagtctactactctttctctctagacccctcacaggtacaagtgtcatcagaaagaggtttaagaatactgagaaatcactcccttcaaagcctagggctgaagagccttagagcactgggagctcatgtactgctccatagacccagggccacatggaatcaatgcagccctcttaggtttctcgagggtgacaggctccagagccacatttctcagaatctgcaatgctgaagattgctctgaatgctggctacttgaagaaatccccggaaaagcttactggcttggaaagagtgacacacctgacagcatgttacaataaaaattttggctgggacactgaggtagcccgttttacaacaaggtgaattttgaagtcgatccccctggagggtttgtgcccatcctaaagacaaagcccaaccacgttgcaccagaaatcactccgctggctcccccagagagtgcacggaggcttcctcctacatggtagaactcaggcaacactcgagcatccgtttaccattgaaggcggctgcatcagtgggaatttccatggcttttcatcccagcatcgaagcgtgtagatgtctggccacagactccttaccttgttcggaaggagcctctaggcttggaaccggggtaacagtcggaggcgcgacggcagtcccttctgcgtctgagcattgcgtcaggttgcagtactcccacctgacaccgggatccctcgtataacaataaggagctgccacagcatctggattcctgcagtagttcatgatcaagccactggaaattccaaaacgatacacgtcacaagaggtgggacaacatgcaggggcaccccacactctctcctttgtgctgcaacaaggtcattaccggtgcctttctaatattcccattaaaagtaccatatgattgccaccagcaaaaatggctctcgatcactaatcctctctgcacatctctcatacttaatagattattactattttttttaaataaaaaatctaaagtagcaaacgcttcttagaaacactgagataacacacacacgtggccgaaaaacgatcagagtattctccttctgccttctgcccaatccatctctctggaataactggtatgggttttgacgtctgtattgtgggattgtgtattca